(19) United States
(12) Patent Application Publication
(10) Pub. No.: US 2026/0204356 A1
(43) Pub. Date: Jul. 16, 2026

(54) METHODS AND SYSTEMS FOR GENERATING PEPTIDES

(71) Applicant: Australian National University, Acton (AU)

(72) Inventors: Matthew Arthur SPENCE, Kaleen (AU); Colin John JACKSON, Kingston (AU); Dana Suzanne MATTHEWS, Ainslie (AU)

(21) Appl. No.: 19/136,834

(22) PCT Filed: Dec. 6, 2023
(86) PCT No.: PCT/IB2023/000741

§ 371 Date: Jun. 6, 2025

Related U.S. Application Data
(60) Provisional application No. 63/431,258, filed on Dec. 8, 2022.
(60) Provisional application No. 63/587,693, filed on Oct. 3, 2023.

(51) Int. Cl. G16B 40/20; G16B 30/10; G16B 40/30

(52) U.S. Cl. CPC G16B 40/20; G16B 30/10; G16B 40/30

(57) ABSTRACT

The present disclosure provides systems and methods related to training a first machine learning model, comprising obtaining the first machine learning model which may comprise a first set of parameters. A first plurality of protein sequences may be obtained. The first plurality of protein sequences may be generated in part by applying an algorithm to at least one test protein sequence. The at least one test protein sequence may comprise at least one masked portion. The first machine learning model may be applied the first plurality of protein sequences to generate a second plurality of protein sequences. At least one protein sequence of the second plurality of protein sequences maybe derived from another protein sequence of the first plurality of protein sequences. At least a subset of the second plurality of protein sequences may be used to adjust the first set of parameters, training the first machine learning model.

### CROSS-REFERENCE

This application claims the benefit of U.S. Provisional Application Ser. No. 63/431,258, filed Dec. 8, 2022, and U.S. Provisional Application Ser. No. 63/587,693, filed Oct. 3, 2023, which are incorporated herewith in entirety.

### BACKGROUND

For any single sequence (e.g., protein or polypeptide sequence), there can be millions of variant sequences. Many of these variants can be useful for purposes that the original sequence was not useful for, and thus, the original sequence is not always the most valuable sequence to have. However, determining all or even a significant portion of possible variant sequences can take extensive time and resources and is currently unable to feasibly be accomplished.

### SUMMARY

An aspect of the disclosure herein describes methods of training a first machine learning model, the method comprising: (a) obtaining the first machine learning model, wherein the first machine learning model comprises a first set of parameters; (b) obtaining a first plurality of biological sequences, wherein the first plurality of biological sequences is generated at least in part by applying an algorithm to at least one test biological sequence, wherein the at least one test biological sequence comprises at least one masked portion; (c) applying the first machine learning model to the first plurality of biological sequences to generate a second plurality of biological sequences, wherein at least one biological sequence of the second plurality of biological sequences is derived from another biological sequence of the first plurality of biological sequences; and (d) using at least a subset of the second plurality of biological sequences to adjust the first set of parameters, thereby training the first machine learning model. In some embodiments, wherein the algorithm is applied by a second machine learning model. In some embodiments, the method further comprises using at least a subset of the first plurality of biological sequences to adjust a second set of parameters of the second machine learning model, thereby training the second machine learning model. In some embodiments, the algorithm is a statistical algorithm. In some embodiments, the statistical algorithm is a Bayesian algorithm or a maximum likelihood algorithm or a maximum parsimony algorithm. In some embodiments, the method further comprises receiving user input received from a device associated with a user; and based at least in part on the received user input, applying a third machine learning model to the second plurality of biological sequences to generate a third plurality of biological sequences. In some embodiments, the user input comprises at least one of: a sequence redundancy threshold; or one or more biological sequences. In some embodiments, the first machine learning model generates the second plurality of biological sequences based at least in part on: a first list containing a distribution of filtered sequence lengths of the second plurality of biological sequences per percent identity redundancy based on a redundancy threshold; or a second list containing a number of biological sequences of the second plurality of biological sequences per percent identity based on the redundancy threshold. In some embodiments, the method further comprises storing the first plurality of biological sequences, the second plurality of biological sequences, or the third plurality of biological sequences in a database. In some embodiments, the method further comprises displaying the first plurality of biological sequences, the second plurality of biological sequences, or the third plurality of biological sequences. In some embodiments, the at least one biological sequence of the second plurality of biological sequences that is derived from another biological sequence of the first plurality of biological sequences, comprises a first biological sequence of the second plurality of biological sequences corresponding to a second biological sequence of the first plurality of biological sequences, wherein the first biological sequence is a modification of the second biological sequence with one or more evolutions. In some embodiments, the one or more evolutions comprise at least one addition, deletion, or substitution. In some embodiments, the second plurality of biological sequences comprises: a plurality of accession numbers corresponding to the sequences of the second plurality of biological sequences; a plurality of accession numbers associated with experimental validation, wherein the plurality of accession numbers corresponds to a first subset of the second plurality of biological sequences; or a plurality of accession numbers associated with solved crystal structures, wherein the plurality of accession numbers corresponds to a second subset of the second plurality of biological sequences. In some embodiments, the third plurality of biological sequences comprises a first subset of the first plurality of biological sequences and a second subset of the second plurality of biological sequences. In some embodiments, the third plurality of biological sequences has fewer sequences than the second plurality of biological sequences. In some embodiments, the redundancy threshold is about 50% to about 99%. In some embodiments, the redundancy threshold is about 50%. In some embodiments, the redundancy threshold is about 70% to about 99%. In some embodiments, the redundancy threshold is about 70%. In some embodiments, the redundancy threshold is about 90%, 91%, 92%, 93%, 94%, 95%, 96%, 97%, 98%, or 99%. In some embodiments, the second machine learning model comprises an Ancestral Sequence Reconstruction (ASR) model. In some embodiments, the first machine learning model comprises a model with parameters trained on biological sequences. In some embodiments, the first machine learning model comprises a transformer model. In some embodiments, the first machine learning model comprises a long short-term memory (LSTM) recurrent neural network model. In some embodiments, the first plurality of biological sequences comprises at least ten thousand different biological sequences. In some embodiments, the method further comprises, prior to (c), subsampling the first plurality of biological sequences to have no more than five hundred different biological sequences. In some embodiments, the second machine learning model generates the first plurality of sequences determining a plurality of biological sequence subsets based on the at least one masked portion, wherein the plurality of biological sequences subsets comprise variations of the at least one masked portion. In some embodiments, the third plurality of sequences does not comprise biological sequences that do not surpass the sequence redundancy threshold, wherein the sequence redundancy threshold is relative to the one or more biological sequences. In some embodiments, the one or more biological sequences comprises the at least one test biological sequence. In some embodiments, the second machine learning model generates the first plurality of sequences using one or more weights. In some embodiments, the method further comprises (e) applying a third machine learning model to the first plurality of biological sequences to optimize the first plurality sequences based on one or more indications. In some embodiments, the one or more indications comprises: robustness of each biological sequence of the first plurality of sequences; stability of each biological sequence of the first plurality of sequences; activity of each biological sequence of the first plurality of sequences; percent identity of each biological sequence of the first plurality of sequences to a selected biological sequence; recombinant expression yield of each biological sequence of the first plurality of sequences; or combinations thereof. In some embodiments, optimizing the first plurality of biological sequences comprises one or more of: removing one or more biological sequences from the first plurality of biological sequences based on one or more indications; adding one or more biological sequences from the first plurality of biological sequences based on the one or more indications; removing one or more portions of one or more biological sequences from the first plurality of biological sequences based on the one or more indications; adding one or more portions of one or more biological sequences of the first plurality of biological sequences based on the one or more indications; or combinations thereof. In some embodiments, the first machine learning model generates subsequent pluralities of biological sequences with one or more of greater accuracy, greater efficiency, or fewer resources relative to machine learning models that are not trained by steps (a) through (d). In some embodiments, the one or more of greater accuracy, greater efficiency, or fewer resources is caused at least in part by the training the first machine learning model using the first plurality of sequences generated by applying the second machine learning model or statistical algorithm. In some embodiments, wherein at least one biological sequence of the first plurality of biological sequences has one or more of an increased robustness, stability, activity, or expression relative to the at least one test biological sequence.

An aspect of the disclosure herein describes methods for generating a plurality of biological sequences, comprising: receiving at least one biological sequence; generating a first plurality of sequences by applying an algorithm to the at least one test biological sequence, wherein the at least one test biological sequence comprises at least one masked portion during the generating of the first plurality of sequences; generating, using a first machine learning model, a second plurality of sequences based on the first plurality of sequences wherein at least one biological sequence of the second plurality of biological sequences is derived from another biological sequence of the first plurality of biological sequences; refining the second plurality of sequences based on one or more parameters to generate a third plurality of sequences; and providing the third plurality of sequences. In some embodiments, the algorithm is applied by a second machine learning model. In some embodiments, the method further comprises receiving feedback based on the third plurality of biological sequences; and training the first machine learning model based on the feedback. In some embodiments, the algorithm is a statistical algorithm. In some embodiments, the statistical algorithm is a Bayesian algorithm or a maximum likelihood algorithm or a maximum parsimony algorithm. In some embodiments, the one or more parameters are received from a device associated with a user. In some embodiments, the one or more parameters comprises at least one of: a sequence redundancy threshold; or one or more biological sequences. In some embodiments, the first machine learning model generates the second plurality of biological sequences based at least in part on: a first list containing a distribution of filtered sequence lengths of the second plurality of biological sequences per percent identity redundancy based on a redundancy threshold; or a second list containing a number of biological sequences of the second plurality of biological sequences per percent identity based on the redundancy threshold. In some embodiments, the method further comprises storing the first plurality of biological sequences, the second plurality of biological sequences, or the third plurality of biological sequences in a database. In some embodiments, the method further comprises displaying the third plurality of biological sequences. In some embodiments, the at least one biological sequence of the second plurality of biological sequences that is derived from another biological sequence of the first plurality of biological sequences, comprises a first biological sequence of the second plurality of biological sequences corresponding to a second biological sequence of the first plurality of biological sequences, wherein the first biological sequence is a modification of the second biological sequence with one or more evolutions. In some embodiments, the one or more evolutions comprise at least one addition, deletion, or substitution. In some embodiments, the second plurality of biological sequences comprises: a plurality of accession numbers corresponding to the sequences of the second plurality of biological sequences; a plurality of accession numbers associated with experimental validation, wherein the plurality of accession numbers corresponds to a first subset of the second plurality of biological sequences; or a plurality of accession numbers associated with solved crystal structures, wherein the plurality of accession numbers corresponds to a second subset of the second plurality of biological sequences. In some embodiments, the third plurality of biological sequences comprises a first subset of the first plurality of biological sequences and a second subset of the second plurality of biological sequences. In some embodiments, the third plurality of biological sequences has fewer sequences than the second plurality of biological sequences. In some embodiments, the redundancy threshold is about 50% to about 99%. In some embodiments, the redundancy threshold is about 50%. In some embodiments, the redundancy threshold is about 70% to about 99%. In some embodiments, the redundancy threshold is about 70%. In some embodiments, the redundancy threshold is about 90%, 91%, 92%, 93%, 94%, 95%, 96%, 97%, 98%, or 99%. In some embodiments, the second machine learning model comprises an Ancestral Sequence Reconstruction (ASR) model. In some embodiments, the first machine learning model comprises a model with parameters trained on biological sequences. In some embodiments, the first machine learning model comprises a transformer model. In some embodiments, the first machine learning model comprises a long short-term memory (LSTM) recurrent neural network model. In some embodiments, the first plurality of biological sequences comprises at least ten thousand different biological sequences. In some embodiments, generating the first plurality of sequences comprises subsampling the first plurality of biological sequences to have no more than five hundred different biological sequences. In some embodiments, the second machine learning model generates the first plurality of sequences by determining a plurality of biological sequence subsets based on the at least one masked portion, wherein the plurality of biological sequences subsets comprise variations of the at least one masked portion. In some embodiments, the third plurality of sequences does not comprise biological sequences that do not surpass the sequence redundancy threshold, wherein the sequence redundancy threshold is relative to the one or more biological sequences. In some embodiments, the one or more biological sequences comprises the at least one test biological sequence. In some embodiments, the second machine learning model generates the first plurality of sequences using one or more weights. In some embodiments, refining the second plurality of sequences based on one or more parameters to generate a third plurality of sequences comprises applying a third machine learning model to the second plurality of biological sequences to optimize the second plurality sequences based on one or more indications. In some embodiments, the one or more indications comprises: robustness of each biological sequence of the first plurality of sequences; stability of each biological sequence of the first plurality of sequences; percent identity of each biological sequence of the first plurality of sequences to a selected biological sequence; activity of each biological sequence of the first plurality of sequences; recombinant expression yield of each biological sequence of the first plurality of sequences; or combinations thereof. In some embodiments, optimizing the first plurality of biological sequences comprises one or more of: removing one or more biological sequences from the first plurality of biological sequences based on one or more indications; adding one or more biological sequences from the first plurality of biological sequences based on the one or more indications; removing one or more portions of one or more biological sequences from the first plurality of biological sequences based on the one or more indications; adding one or more portions of one or more biological sequences of the first plurality of biological sequences based on the one or more indications; or combinations thereof. In some embodiments, the first machine learning model generates subsequent pluralities of biological sequences with one or more of greater accuracy, greater efficiency, or fewer resources relative to machine learning models that are not trained by steps (a) through (d). In some embodiments, the one or more of greater accuracy, greater efficiency, or fewer resources is caused at least in part by the training the first machine learning model using the first plurality of sequences generated by applying the second machine learning model. In some embodiments, at least one biological sequence of the first plurality of biological sequences has one or more of an increased robustness, stability, activity, or expression relative to the at least one test biological sequence.

Another aspect of the present disclosure provides a non-transitory computer-readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the subject matter described herein are set forth with particularity in the appended claims. A better understanding of the features and advantages of the present subject matter will be obtained by reference to the following detailed description that sets forth illustrative embodiments, in which the principles of the present subject matter are utilized, and the accompanying drawings (also “Figure” and “FIG.” herein), of which:

FIG. 1 depicts an exemplary system for training a machine learning model.

FIG. 2 depicts an example process for generating a plurality of result sequences.

FIG. 3 depicts an example process for generating a plurality of reconstructed sequences using ancestral reconstruction.

FIG. 4 depicts an example process for generating a plurality of evolved sequences.

FIG. 5 depicts an example process for generating a plurality of result sequences.

FIG. 6 depicts an example method for training a first machine learning model.

FIG. 7 depicts an example method for run-time processes of generating a plurality of result sequences based on one or more test sequences.

FIG. 8 depicts a computer system that is programmed or otherwise configured to implement methods provided herein.

FIG. 9 depicts one or more example processing devices in communication with one or more other processing devices.

FIG. 10 depicts one or more example processing devices in communication with a network.

FIG. 11 depicts the performance of specific models versus the number of parameters for LASR embeddings as compared to previously used models.

FIG. 12 depicts wall-time in logarithmic minutes for the for the in silico evolution of PTE using ESM-1b and LASR as embedding schemes.

FIGS. 13A-13C depict sequence representations projected onto a 2-dimensional t-distributed stochastic neighbor embedding.

FIGS. 14A-14C depict trajectories and mutants for sequence embeddings, as well as hamming distances between sequences and parents of those sequences, and catalytic efficiency for generated sequences.

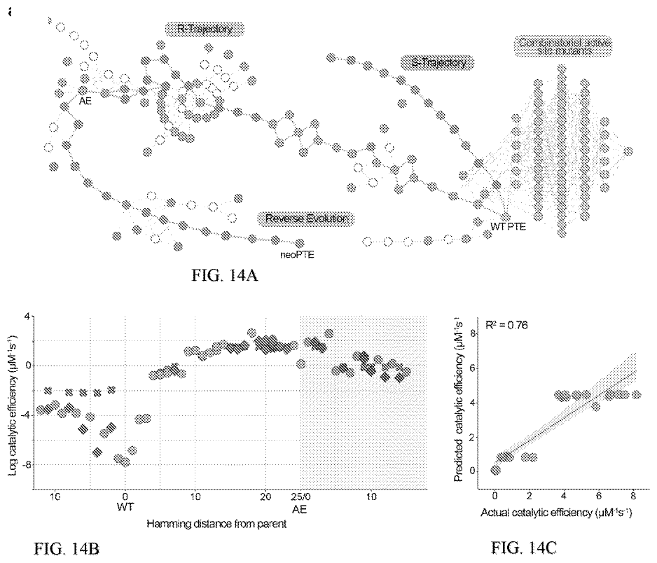

FIGS. 15A-15D depict the Dirichlet energy for sequences as well as trajectories of processes associated with those sequences.

FIGS. 16A-16F depict relative fitnesses, Dirichlet energy, and Fourier Coefficients for representation spaces for a representation model trained on sequence data produced with ASR.

### DETAILED DESCRIPTION

Obtaining the exact sequence for a particular purpose can often be an extremely difficult task if either the exact sequence, or the particular purpose, are not known. For example, a given sequence for a given first purpose may be known, and it may be inferred that the given sequence may be modified to fulfill a specific second purpose. However, if the given sequence is even 100 amino acids long, determining all possible modifications to the given sequence, and subsequently, choosing which of the modifications that will lead to the appropriate sequence for fulling the given second purpose is incredibly difficult, resource-intensive, and time-consuming due to the innumerable possibilities, even if extra knowledge allows for the pool of possible sequences to be narrowed. Thus, there is a need in the art to be able to determine what sequences fulfill desired purposes without the difficulty and requirements of conventional methods.

Systems and methods described herein address the problems faced by conventional methods by significantly reducing the resources and time required to determine a sequence out of a pool of possible protein modifications that could fulfill a desired purpose based on a sample sequence (e.g., a protein sequence or nucleotide sequence). By performing ancestral reconstruction on the sample sequence, many more sequences that are related to the sample sequence are determined. Further, those numerous sequences may then be modified or evolved in a multitude of ways, creating a larger set of sequences that are related to the original sequence. A machine learning model can be trained to evolve the numerous sequences in ways that allow for the best sequences to be generated—for example, sequences with greater activity than the sample sequence. In some cases, the machine learning model could instead be trained to evolve the numerous sequences in ways that allow sequences with lower activity than the sample sequence, depending on what the ideal sequence would be used for. With the evolution of the sequences, the most ideal sequences can be generated, rather than all possible sequences, which lowers the amount of required resources and time needed to generate the sequences. Even further, the sequences can then be narrowed down as necessary by using certain criteria, such as sequence alignment with the sample sequence, further reducing the amount of resources and time necessary to go review and test the resulting sequences to determine which are best suited for the desired purpose.

Thus, by performing ancestral reconstruction on the sample sequence, training a machine learning model to evolve the sequences to generate ideal sequences, using the model to evolve the reconstructed sequences to generate the ideal sequences, and then further narrowing those sequences down so that only the desired sequences remain, the system and methods described herein increase the efficiency and decrease the amount of resources that would be required by conventional methods of determining an ideal sequence for the desire purpose.

### Representation Learning and Ancestral Sequence Reconstruction

Protein representation learning has been transformative for protein engineering and evolutionary inference 1-5. Representation learning aims to transform discrete protein sequences, which are inherently high-dimensional and information sparse, into dense vector representations that numerically capture the salient features of the protein sequence. These representations capture the complex relationships between amino acids and provide insights into the functional characteristics and evolutionary histories of the proteins that are embedded in the representation mode. Representations are often provided by protein language models (PLMs) that are trained on large databases of protein sequences. To build representations that capture a protein's biophysical and evolutionary features in the absence of a functional label, Protein language models (PLMs) are often trained by unsupervised masked language modeling (MLM), in which the model is tasked with predicting the identities of residues that have been masked from the surrounding sequence context. The hidden states of a trained PLM, which are retrieved and pooled into a representation, implicitly capture the relevant physical and biological properties of protein sequences. Protein representations can also be derived empirically, such as from the orthogonal principal components of physical amino acid descriptors (such as hydrophobicity, solvent accessible surface area, charge); however, such representations fail to capture the context-dependence that deep representation models, such as PLMs, do. This imbues deep representation models, such as PLMs, a context awareness that empirically derived representations, such as from the orthogonal principal components of physical amino acid descriptors fail to capture, drastically improving the accuracy/usefulness/validity of their insights.

Protein representation models project sequences to a semantically-rich embedding space that supervised learning models can leverage to excel at supervised tasks (e.g., fitness prediction). This helps overcome the inherent challenges of supervised learning in an information-sparse discrete sequence domain confounded by epistasis (i.e., non-linearity in the fitness landscape). Additionally, the phenomenon of epistasis is synonymous with ruggedness in the fitness landscape. For example, a highly epistatic system is one in which the effect of a mutation is highly contingent on the background into which it is introduced. In highly rugged or epistatic fitness landscapes, a small number of mutations can lead to dramatic, non-linear changes in the observed fitness, making prediction of the functional outcomes of mutations fundamentally challenging. As a result, high-order epistasis renders molecular evolutionary trajectories unpredictable when amino acids are encoded by their discrete character identity. Recent work has shown that deep neural networks that add explicit regularization to smooth the model's latent space, and those that account for epistasis coefficients, can significantly improve the utility of representations at downstream supervised tasks. Accordingly, smooth embedding spaces with respect to the observed protein function may be more informative than those which are not. In the case of unsupervised PLMs, implicit smoothing in the representation space may occur via the learning of contextual sequence dependencies from the MLM objective; therefore, training on sequence data that maximizes the model's predictive comprehension of epistasis (i.e. contextual sequence dependencies) may produce more informative protein representations.

Additionally, ancestral sequence reconstruction (ASR) is a statistical method used to infer extinct molecular sequences of a phylogenetic tree. Sequences generated by ASR are functionally enriched and often feature novel phenotypes or properties that are ideal for protein engineering. Accordingly ASR has many uses, such as determining protein thermostabilization, functional sequence exploration, and the generating novel protein scaffolds. Even further, ASR outperforms state-of-the-art deep neural machines, including large PLMs, at functional protein generation and has also provided tremendous insight into molecular evolutionary processes and understanding sequence-function relationships, which allows for studying the biophysical, chemical, and biological properties of extinct ancestral sequences can reveal the mechanisms by which proteins acquire novel phenotypes and functions, as well as the sequence features, such as epistasis, that either confound or enable them.

Accordingly, the systems and methods described herein use sequences generated through ASR to train family-specific protein representations model in order to improve both the effectiveness and efficiency in predicting properties of sequences, such as the activity of a sequence.

### Example System for Training One or More Machine Learning Models to Generate Result Sequences

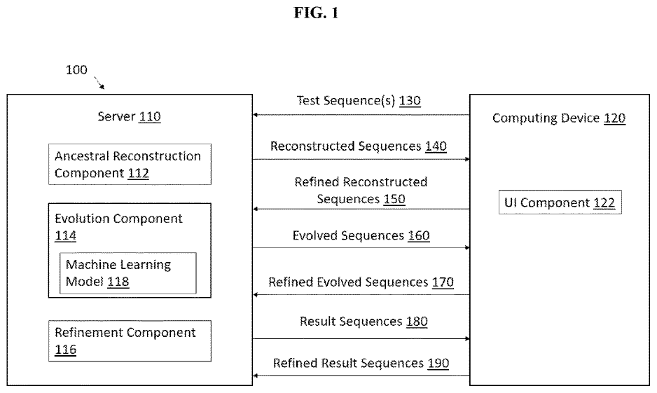

FIG. 1 depicts an example system 100 for training one or more machine learning models to generate a plurality of biological sequences (e.g., “representations”, also referred to as “embeddings”, of amino acid/protein sequences and/or nucleotide sequences). In this depicted example, system 100 comprises a server 110 and a computing device 120. In some embodiments, the system 100 does not include the computing device 120.

In this depicted embodiment, server 110 further includes ancestral reconstruction component 112, evolution component 114, and refinement component 116.

Ancestral reconstruction component 112 may generate a plurality of ancestrally reconstructed sequences (also referred to herein as “reconstructed sequences”) from one or more sequences. In some embodiments, the one or more sequences and the sequences of the plurality of reconstructed sequences are amino acid/protein sequences or nucleotide sequences. In this depicted embodiment, the one or more sequences are test sequence(s) 130, which are received by ancestral component 112. In this depicted embodiment, the test sequences(s) 130 are received from the component device 120. In other embodiments, the ancestral reconstruction component may receive the one or more sequences by a different method, such as retrieving the one or more sequences from a database on the server.

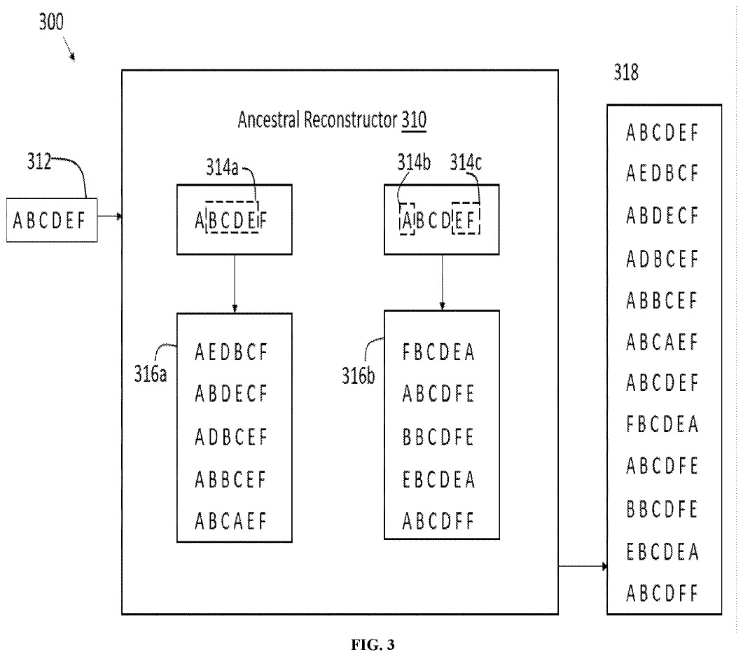

Ancestral reconstruction component 112 may be configured to perform ancestral reconstruction on the one or more sequences (e.g., test sequence(s) 130) in order to generate a plurality of reconstructed sequences 140 (e.g., by generating sequences based on masking one or more portions of sequences or an entire sequence, as described further below with respect to FIG. 3). Ancestral reconstruction is defined as “phylogenetic inference of ancient sequences, followed by gene synthesis, expression, and experimental characterization”—and is a widely used strategy to experimentally test hypotheses about the functional and biochemical properties of ancient proteins. Using ancestral reconstruction allows for the inference of related and/or useful sequences. Ancestral reconstruction component 112 may perform ancestral reconstruction on the test sequence(s) using one or more methods. In some embodiments, the one or more methods include utilizing a machine learning model to generate a plurality of reconstructed sequences 140. In other embodiments, the one or more methods include using an algorithm to generate the plurality of reconstructed sequences 140. In some embodiments, the algorithm may be a statistical algorithm. In some embodiments, the statistical algorithm may be a maximum likelihood ancestral sequence reconstruction, empirical Bayesian ancestral sequence reconstruction, or hierarchical Bayesian ancestral sequence reconstruction or maximum parsimony ancestral sequence reconstruction. While some statistical algorithms are listed, they are exemplary, and others may be used.

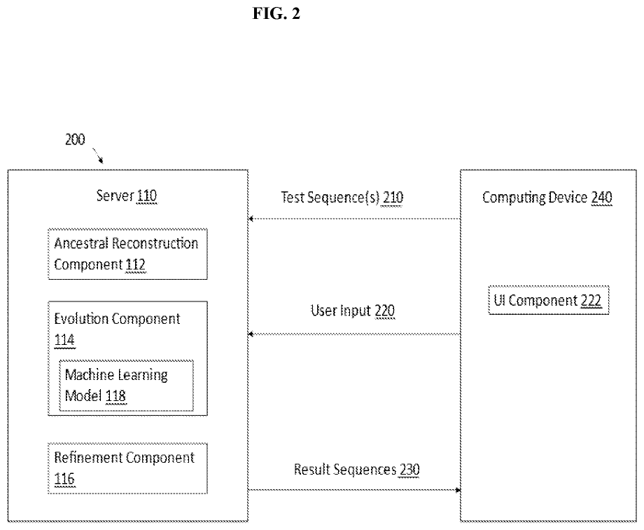

In some embodiments, the algorithm may be applied by a reconstruction machine learning model of the ancestral reconstruction component 112. In some embodiments, the reconstruction machine learning model may be previously trained generate the plurality of reconstructed sequences using at least one sequence as input. In some embodiments, the machine learning model may be previously trained on the server 110 or on a different processing device. In those embodiments, the reconstruction machine learning model is trained to generate the plurality of reconstructed sequences using the test sequence(s) 130 as input. For example, the reconstruction machine learning model may receive the test sequence(s) and generate (e.g., predict) the plurality of reconstructed sequences 140 (as further described below with respect to FIG. 2). In some embodiments, generating (e.g., predicting) the plurality of reconstructed sequences may include generating the reconstructed sequences based on the test sequence(s) 130. In some embodiments, generating (e.g., predicting) the plurality of reconstructed sequences 140 may include generating and/or outputting representations of additions, deletions, or substitutions to the test sequence(s) 130 that result in the plurality of reconstructed sequences 140. In those embodiments, the generated representations may include values associated with one or more changes to the test sequence(s) 130 that can be used to indicate the plurality of reconstructed sequences 140 (e.g., values indicating differences between the test sequence(s) 130 and each of the plurality of reconstructed sequences 140). The reconstruction machine learning model may further generate the reconstructed sequences 140 using one or more parameters. In some embodiments, the reconstructed sequences 140 include no more than five hundred different sequences, but can include thousands of different sequences (e.g., ten thousand different sequences).

The ancestral reconstruction component 112 may further generate the reconstructed sequences 140 based one or more stored confident sequences, wherein the one or more stored confident sequences are associated with reconstructed sequences associated with the test sequence(s) 130. In some embodiments, the stored confident sequences may have been previously generated by the ancestral reconstruction component 112 or evolution component 114. In some embodiments, those previously generated confident sequences may have been approved as confident sequences and stored by machine learning model. In some embodiments, the confident sequences are received and stored by ancestral reconstruction component 112.

Ancestral reconstruction component 112 may further be configured to send the plurality of reconstructed sequences 140 for use in generating result sequences. In this depicted example, ancestral component 112 sends the plurality of reconstructed sequences 140 to computing device 120. In some embodiments, the server 110 receives feedback regarding the reconstructed sequences 140. In some embodiments, the feedback may be provided by computing device 120. In this depicted example, the server 110 receives a plurality of refined reconstructed sequences 150 as feedback. In some embodiments, the refined reconstructed sequences 150 may include one or more of the plurality of reconstructed sequences 140. In some embodiments, the refined reconstructed sequences 150 may not include all of the plurality of reconstructed sequences 140. In some embodiments, the refined reconstructed sequences 150 includes one or more variants of one or more sequences of the plurality of reconstructed sequences 140. In some embodiments, the plurality of refined reconstructed sequences 150 includes a first number of sequences and the plurality of reconstructed sequences 150 includes a second number of sequences, wherein the first number of sequences is lower than the second number of sequences. In the embodiments where feedback is received, the feedback is used by the reconstruction machine learning model to refine the process by which the reconstruction machine learning model generates the plurality of reconstructed sequences (e.g., by adjusting one or more weights so that the reconstruction machine learning model generates refined reconstructed sequences when receiving test sequence(s) as input and/or storing one or more sequences of the plurality of refined reconstructed sequences 150), thereby training the reconstruction machine learning model. In some embodiments, the reconstruction machine learning model may be an Ancestral Data Reconstruction (“ASR”) model. In some embodiments, a combination of previously generated pluralities of sequences may be combined into one or more pluralities of sequences. In some embodiments, the plurality of reconstructed sequences is generated using statistical reconstruction.

In this depicted embodiment, evolution component 114 may be configured to generate a plurality of evolved sequences 160 using evolution component 114. In this depicted example, the evolution component 114 includes machine learning model 118 (also referred to herein as the “evolution machine learning model”). In some embodiments, the machine learning model 118 may be trained from parameters initialized from a model previously trained on protein and/or nucleotide sequences, for example a unified representation model (“UniRep”) model. In some embodiments, the machine learning model 118 is a transformer model. In some embodiments, the machine learning model 118 is a long short-term memory (“LSTM”) recurrent neural network model. In some embodiments, the machine learning model 118 is a recurrent neural network (“RNN”). In some embodiments, the machine learning model 118 is a transformer model. In some embodiments, the machine learning model 118 is a convolutional neural network (“CNN”). In some embodiments, the machine learning model 118 is a Learned Ancestral Sequence Reconstruction (“LASR”) model initialized with random parameters. In this depicted example, the machine learning model 118 receives the plurality of refined reconstructed sequences 150 and evolves one or more sequences of the plurality of refined reconstructed sequences 150 to generate a plurality of evolved sequences 160. The machine learning model 118 may evolve the one or more sequences of the plurality of refined sequences by modifying the one or more sequences of the plurality of refined sequences to generate an evolved sequence. In some embodiments, modifying the one or more sequences includes adding or removing at least one amino acid from the one or more sequences. In some embodiments, modifying the one or more sequences includes substituting at least one amino acid from the one or more sequences. In some embodiments, the machine learning model 118 may further generate the plurality of evolved sequences 160 based on a first list containing a distribution of filtered sequence lengths of the second plurality of sequences per percent identity redundancy based on a redundancy threshold or a second list containing a number of sequences of the evolved plurality of sequences 160 per percent identity based on the redundancy threshold. For example, the evolution component may not generate sequences less than filtered sequence lengths or may only include or exclude sequences based on the percent identity redundancy. In this depicted embodiment, the plurality of evolved sequences 160 generated by evolution component 114 includes both the evolved sequences generated by machine learning model 118 and the plurality of refined reconstructed sequences 150. In some embodiments, the plurality of evolved sequences 160 generated by evolution component 114 includes only the evolved sequences generated by machine learning model 118. In some embodiments, the plurality of evolved sequences 160 also includes a plurality of accession numbers corresponding to the sequences of the plurality of evolved sequences 160, a plurality of accession numbers associated with experimental validation, wherein the plurality of accession numbers corresponds to a first subset of the plurality of evolved sequences 160, a plurality of accession numbers associated with solved crystal structures, wherein the plurality of accession numbers corresponds to a second subset of sequences of the plurality of evolved sequences 160, or combinations thereof.

The evolution component 114 may be configured to send the plurality of evolved sequences 160 for generating use in generating result sequences. In this depicted example, evolution component 114 provides the plurality of evolved sequences 160 to computing device 120. In other embodiments, the evolution component 114 may send the plurality of evolved sequences 160 to another processing device. In some embodiments, the evolution component 114 may not provide the plurality of evolved sequences 160 to another processing device. In some embodiments, the evolution component 114 may provide the plurality of evolved sequences 160 to another component of the server 110. In some embodiments, the evolution component 114 may receive feedback regarding the plurality of evolved sequences 160. In this depicted embodiment, the received feedback is the plurality of refined evolved sequences 170. In this depicted embodiment, the plurality of refined evolved sequences 170 includes one or more of the plurality of evolved sequences 160. In some embodiments, the plurality of refined evolved sequences 170 does not include all of the plurality of evolved sequences 160. In some embodiments, the refined plurality of evolved sequences 170 includes one or more variants of one or more sequences of the plurality of evolved sequences 160. In some embodiments, the plurality of refined evolved sequences 170 includes a third number of sequences and the plurality of evolved sequences 160 includes a fourth number of sequences, where the third number is lower than the fourth number of sequences. In the embodiments where feedback is received, the feedback may be used by the evolution component 114 to refine the process by which the evolution component 114 and machine learning model 118 generates the plurality of evolved sequences 160 (e.g., by adjusting one or more weights so that the machine learning model 118 generates refined evolved sequences when receiving a plurality of evolved sequences and/or storing one or more sequences as confident sequences for use by ancestral reconstruction component 112 or evolution component 114), thereby training the machine learning model 118.

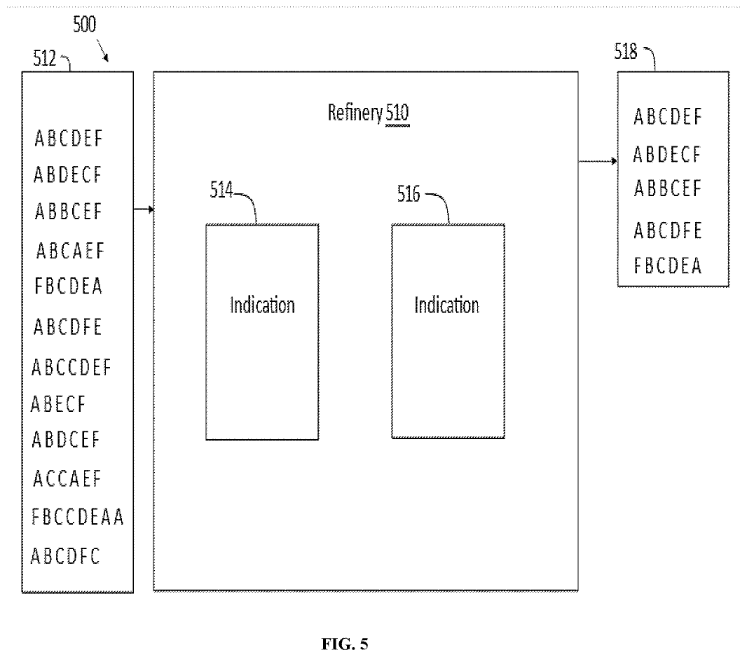

In this depicted embodiment, the refinement component 116 may be configured to receive the plurality of refined evolved sequences 170. Refinement component 118 may further be configured to perform refinement of the plurality of refined evolved sequences 170 (as further described with respect to FIG. 5). For example, refinement component 116 may receive the plurality of refined evolved sequences 170 as input and generate a plurality of result sequences 180 using one or more methods. In some embodiments, the one or more methods may include utilizing a machine learning model (hereinafter also referred to as the refinement machine learning model) to generate the plurality of result sequences 180. In other embodiments, the one or more methods may include utilizing an algorithm to generate the plurality of result sequences 180 based on the plurality of refined evolved sequences 170 and user input from an associated computing device. The user input may include one or more of a percent identity threshold to one or more selected sequences, the robustness of one or more sequences, the stability of one or more sequences, an activity of one or more sequences, an expression of one or more sequences, or combinations thereof. The robustness of a generated sequence may be defined as how much the generated sequence resemble and behave like a natural protein. In some embodiments, the percent identity threshold may be about 50%, about 55%, about 60%, about 65%, about 70%, about 75%, about 80%, about 85%, about 90%, about 95%, about 96%, about 97%, about 98%, or about 99%. In some embodiments, the one or more selected sequences may include test sequence(s) 130. The refinement component 116 may be configured to adding or remove sequences or portions of sequences from the plurality of refined evolved sequences 170 based on whether the individual sequences of the plurality of refined evolved sequences 170 satisfy one or more indications. The one or more indications may be stored on server 110. The one or more indications may also be the user input described above.

In some embodiments, the refinement component 116 may include the refinement machine learning model. The refinement machine learning model may be trained to generate a plurality of result sequences 180 based on the plurality of refined evolved sequences 170. For example, the refinement machine learning model may receive the plurality of refined evolved sequences 170 as input and generate the plurality of result sequences 180. In some embodiments, the refinement machine learning model may further generate the plurality of result sequences 180 using one or more parameters. In some embodiments, the refinement machine learning model may further generate the plurality of result sequences 180 based on user input indicating values of one or more indications, as further described with respect to FIG. 5.

In this depicted embodiment, the refinement component 116 may be configured to send the plurality of result sequences 180 to computing device 120. In some embodiments, the refinement component 116 may provide the plurality of result sequences to another processing device. In some embodiments, the server 110 receives feedback regarding the plurality of result sequences 180. In some embodiments, the feedback is provided by computing device 120. In this depicted embodiment, the server 110 is receives the refined result sequences 190 as feedback. The plurality of refined result sequences 190 may include one or more of the plurality of result sequences 180. In some embodiments, the plurality of refined result sequences 190 does not include all of the plurality of result sequences 180 associated with values of the one or more indications. In some embodiments, the plurality of refined result sequences 190 includes a fifth number of sequences and the plurality of result sequences 180 includes a sixth number of sequences, where the fifth number of sequences is lower than the sixth number of sequences. In the embodiments where feedback is received, the feedback is used by the refinement machine learning model to refine the process by which the refinement machine learning model generates the plurality of result sequences (e.g., by adjusting one or more parameters or one or more values of indications used in generating pluralities of result sequences and/or storing one or more sequences of the plurality of refined result sequences 190), thereby training the refinement machine learning model. In some embodiments, the refinement machine learning model may further be trained to predict the values of the one or more indications that is used to generate the plurality of result sequences 180 based on the plurality of refined evolved sequences 170 and/or test sequence(s) 130. In those embodiments, no user input may be required beyond training the refinement machine learning model.

In this depicted embodiment, computing device 120 further comprises user interface (“UI”) component 122. In some embodiments, user input to the user interface component 122 receive user input. User input may include individual sequences or various pluralities of sequences, such as the test sequence(s) 130, the plurality of refined reconstructed sequences 150, the plurality of refined evolved sequences 170, and/or the plurality of refined result sequences 190. In some embodiments, the user input may include editions to the various pluralities of sequences. In those embodiments, the ancestral reconstruction component 112, the evolution component 114, and/or the refinement component 116 may implement the editions to the plurality of reconstructed sequences 140, the plurality of evolved sequences 160, and/or the plurality of result sequences 180, respectively. In some embodiments, any of test sequence(s) 130, one or more sequences of plurality of reconstructed sequences 140, one or more sequences of plurality of refined reconstructed sequences 150, one or more sequences of plurality of evolved sequences 160, one or more sequences of plurality of refined evolved sequences 170, one or more sequences of plurality of result sequences 180, and/or one or more sequences of plurality of refined result sequences 190 may be displayed on UI component 122. Additionally, the UI component 122 may display one or more LASR representations associated with the plurality of reconstructed sequences 140, the plurality of evolved sequences 160, and/or the plurality of result sequences 180, respectively. In some embodiments, any of test sequence(s) 130, one or more sequences of plurality of reconstructed sequences 140, one or more sequences of plurality of refined reconstructed sequences 150, one or more sequences of plurality of evolved sequences 160, one or more sequences of plurality of refined evolved sequences 170, one or more sequences of plurality of result sequences 180, and/or one or more sequences of plurality of refined result sequences 190.

Thus, based on one or more test sequences as well as feedback to the various components of server 110, the system 100 can train one or more machine learning models to generate a desired plurality of result sequences.

In some embodiments, the redundancy threshold may include a range of percentages (e.g., 70% to 99%). In some embodiments, the redundancy threshold may include a value of redundancy that may be met. In some embodiments, the redundancy threshold is about 50% to about 90%. In some embodiments, the redundancy threshold is about 50% to about 55%, about 50% to about 60%, about 50% to about 65%, about 50% to about 70%, about 50% to about 75%, about 50% to about 80%, about 50% to about 85%, about 50% to about 90%, about 55% to about 60%, about 55% to about 65%, about 55% to about 70%, about 55% to about 75%, about 55% to about 80%, about 55% to about 85%, about 55% to about 90%, about 60% to about 65%, about 60% to about 70%, about 60% to about 75%, about 60% to about 80%, about 60% to about 85%, about 60% to about 90%, about 65% to about 70%, about 65% to about 75%, about 65% to about 80%, about 65% to about 85%, about 65% to about 90%, about 70% to about 75%, about 70% to about 80%, about 70% to about 85%, about 70% to about 90%, about 75% to about 80%, about 75% to about 85%, about 75% to about 90%, about 80% to about 85%, about 80% to about 90%, or about 85% to about 90%. In some embodiments, the redundancy threshold is about 50%, about 55%, about 60%, about 65%, about 70%, about 75%, about 80%, about 85%, or about 90%. In some embodiments, the redundancy threshold is at least about 50%, about 55%, about 60%, about 65%, about 70%, about 75%, about 80%, or about 85%. In some embodiments, the redundancy threshold is at most about 55%, about 60%, about 65%, about 70%, about 75%, about 80%, about 85%, or about 90%. In some embodiments, the redundancy threshold is about 70% to about 80%. In some embodiments, the redundancy threshold is about 70% to about 71%, about 70% to about 72%, about 70% to about 73%, about 70% to about 74%, about 70% to about 75%, about 70% to about 76%, about 70% to about 77%, about 70% to about 78%, about 70% to about 79%, about 70% to about 80%, about 71% to about 72%, about 71% to about 73%, about 71% to about 74%, about 71% to about 75%, about 71% to about 76%, about 71% to about 77%, about 71% to about 78%, about 71% to about 79%, about 71% to about 80%, about 72% to about 73%, about 72% to about 74%, about 72% to about 75%, about 72% to about 76%, about 72% to about 77%, about 72% to about 78%, about 72% to about 79%, about 72% to about 80%, about 73% to about 74%, about 73% to about 75%, about 73% to about 76%, about 73% to about 77%, about 73% to about 78%, about 73% to about 79%, about 73% to about 80%, about 74% to about 75%, about 74% to about 76%, about 74% to about 77%, about 74% to about 78%, about 74% to about 79%, about 74% to about 80%, about 75% to about 76%, about 75% to about 77%, about 75% to about 78%, about 75% to about 79%, about 75% to about 80%, about 76% to about 77%, about 76% to about 78%, about 76% to about 79%, about 76% to about 80%, about 77% to about 78%, about 77% to about 79%, about 77% to about 80%, about 78% to about 79%, about 78% to about 80%, or about 79% to about 80%. In some embodiments, the redundancy threshold is about 70%, about 71%, about 72%, about 73%, about 74%, about 75%, about 76%, about 77%, about 78%, about 79%, or about 80%. In some embodiments, the redundancy threshold is at least about 70%, about 71%, about 72%, about 73%, about 74%, about 75%, about 76%, about 77%, about 78%, or about 79%. In some embodiments, the redundancy threshold is at most about 71%, about 72%, about 73%, about 74%, about 75%, about 76%, about 77%, about 78%, about 79%, or about 80%. In some embodiments, the redundancy threshold is about 80% to about 90%. In some embodiments, the redundancy threshold is about 80% to about 81%, about 80% to about 82%, about 80% to about 83%, about 80% to about 84%, about 80% to about 85%, about 80% to about 86%, about 80% to about 87%, about 80% to about 88%, about 80% to about 89%, about 80% to about 90%, about 81% to about 82%, about 81% to about 83%, about 81% to about 84%, about 81% to about 85%, about 81% to about 86%, about 81% to about 87%, about 81% to about 88%, about 81% to about 89%, about 81% to about 90%, about 82% to about 83%, about 82% to about 84%, about 82% to about 85%, about 82% to about 86%, about 82% to about 87%, about 82% to about 88%, about 82% to about 89%, about 82% to about 90%, about 83% to about 84%, about 83% to about 85%, about 83% to about 86%, about 83% to about 87%, about 83% to about 88%, about 83% to about 89%, about 83% to about 90%, about 84% to about 85%, about 84% to about 86%, about 84% to about 87%, about 84% to about 88%, about 84% to about 89%, about 84% to about 90%, about 85% to about 86%, about 85% to about 87%, about 85% to about 88%, about 85% to about 89%, about 85% to about 90%, about 86% to about 87%, about 86% to about 88%, about 86% to about 89%, about 86% to about 90%, about 87% to about 88%, about 87% to about 89%, about 87% to about 90%, about 88% to about 89%, about 88% to about 90%, or about 89% to about 90%. In some embodiments, the redundancy threshold is about 80%, about 81%, about 82%, about 83%, about 84%, about 85%, about 86%, about 87%, about 88%, about 89%, or about 90%. In some embodiments, the redundancy threshold is at least about 80%, about 81%, about 82%, about 83%, about 84%, about 85%, about 86%, about 87%, about 88%, or about 89%. In some embodiments, the redundancy threshold is at most about 81%, about 82%, about 83%, about 84%, about 85%, about 86%, about 87%, about 88%, about 89%, or about 90%. In some embodiments, the redundancy threshold is about 90% to about 99%. In some embodiments, the redundancy threshold is about 90% to about 91%, about 90% to about 92%, about 90% to about 93%, about 90% to about 94%, about 90% to about 95%, about 90% to about 96%, about 90% to about 97%, about 90% to about 98%, about 90% to about 99%, about 91% to about 92%, about 91% to about 93%, about 91% to about 94%, about 91% to about 95%, about 91% to about 96%, about 91% to about 97%, about 91% to about 98%, about 91% to about 99%, about 92% to about 93%, about 92% to about 94%, about 92% to about 95%, about 92% to about 96%, about 92% to about 97%, about 92% to about 98%, about 92% to about 99%, about 93% to about 94%, about 93% to about 95%, about 93% to about 96%, about 93% to about 97%, about 93% to about 98%, about 93% to about 99%, about 94% to about 95%, about 94% to about 96%, about 94% to about 97%, about 94% to about 98%, about 94% to about 99%, about 95% to about 96%, about 95% to about 97%, about 95% to about 98%, about 95% to about 99%, about 96% to about 97%, about 96% to about 98%, about 96% to about 99%, about 97% to about 98%, about 97% to about 99%, or about 98% to about 99%. In some embodiments, the redundancy threshold is about 90%, about 91%, about 92%, about 93%, about 94%, about 95%, about 96%, about 97%, about 98%, or about 99%. In some embodiments, the redundancy threshold is at least about 90%, about 91%, about 92%, about 93%, about 94%, about 95%, about 96%, about 97%, or about 98%. In some embodiments, the redundancy threshold is at most about 91%, about 92%, about 93%, about 94%, about 95%, about 96%, about 97%, about 98%, or about 99%.

### Multiplexed ASR

Training deep representation models can require large sequence datasets. Unlike deep generative models, however, ASR is conditioned on a prior phylogenetic hypothesis that includes a phylogenetic topology and a molecular sequence evolution model. Reconstructed sequences belong to discrete bifurcations in the prior phylogenetic topology and are therefore inherently restrained to the underlying tree structure. From a fully resolved and rooted phylogeny with “n” tips, at most n−1 ancestral sequences can be reconstructed if only the most likely (the maximum a posteriori, MAP) sequence is sampled from the posterior probability distribution of each internal node.

Accordingly, a multiplex ASR (mASR) pipeline may be developed for use in the systems and methods described herein (e.g., through ancestral reconstruction component 112 of FIG. 1) that samples statistically equivalent topologies as priors for ASR. Because the ground-truth phylogeny cannot ever be known with certainty, phylogenetic topologies are reconstructed by heuristic tree-search algorithms that minimize the negative log-likelihood for a particular tree. By performing numerous and independent tree searches in parallel, and filtering those that are not statistically equivalent by the approximately unbiased (AU) test, a pool of equally valid, yet distinct phylogenies may be generated and are used to reconstruct ancestral sequences, as described further above. The vast size of tree space means that identical topologies are seldom returned by random independent tree searches, effectively increasing the number of sequences produced through ASR by a factor equal to the number of tree-search replicates that are accepted by the AU test.

Protein evolution models used in ASR consider only synonymous mutations as part of the molecular evolutionary process. Because insertion/deletion events are often responsible for driving functional diversification, an automated pipeline for the maximum likelihood reconstruction of insertions/deletions in ancestral sequences was also developed. For site “n”, all tips are assigned a binary label (0 for no insertion, 1 for insertion), depending on whether a gap character is observed. A rate of insertion in a sequence that is approximately equal to the rate of deletion may be assumed, and an equal-rates model may be used to reconstruct the probability of a gap being present at site n of all ancestral nodes. Such a process may be repeated for all sites where a gap is observed in >=1% of the extant sequences and remove all residues from ancestral nodes reconstructed with a label of <0.5.

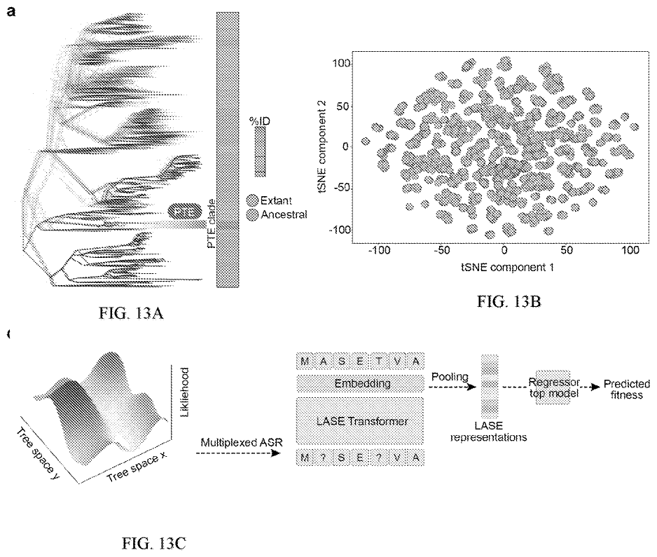

In a specific example, mASR is applied to the xenobiotic degrading enzyme phosphotriesterase (PTE). PTE hydrolyses synthetic phosphotriester pesticides and has been used extensively as a model for functional adaptation and molecular evolution. From an initial alignment of 293 non-redundant, extant sequences, a dataset of >10000 unique and new-to-nature protein sequences was generated with 100 replicates of tree-search, despite most replicates returning highly similar topologies (as described further with respect to Example 8 and FIG. 13A). In this particular example, ancestral and extant sequences were embedded, in the PLM ESM-1b's latent space, which was been pre-trained by a machine learning model (e.g., a machine learning model of ancestral reconstruction component 112) on ~250 million non-redundant protein sequences in the UniRef50-S database. T-distributed stochastic neighbor embedding (tSNE) was used to project the ESM-1b representations of extant and ancestral PTEs onto 2 dimensions for visualization (FIG. 13B). The majority of ancestral sequences produced with mASR belong to regions of sequence space that are not sampled by the extant PTE homologs used to reconstruct them. Accordingly, since ancestrally reconstructed proteins often feature increased thermostabilities with comparable catalytic and biological activities to the extant proteins used to reconstruct them and the majority of the sequences has also not yet been sampled, the produced sequences indicated that mASR may serve as a source of functionally enriched, new-to-nature sequence novelty. Thus, since ancestral sequences are not represented in large, pre-trained pLMs, ancestral-like sequences are highly unlikely to be produced by generative PLMs designed specifically for novel sequence generation in protein engineering, meaning that mASR is a unique and singular method for their production, showing an improvement in discovering novel sequences over conventional methods.

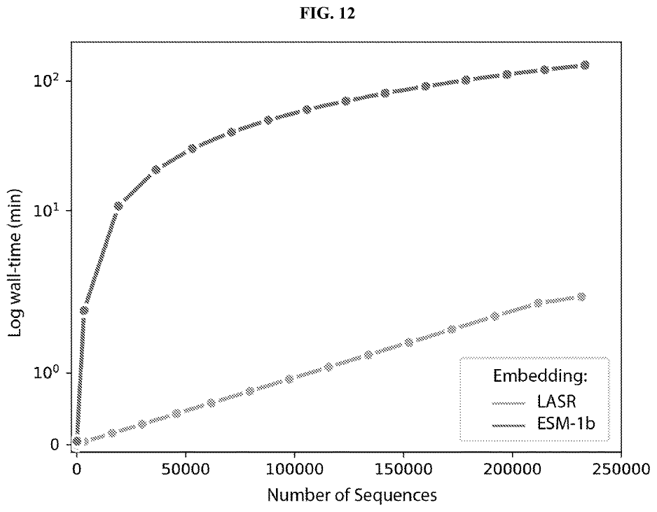

Even further, LASR shows significant improvements over conventional methods through increased inference speed. For example, in silico evolution was performed in a discrete sequence domain to quantify the wall-time speed-up that LASR provides relative to the next best performing representation model, ESM-1b. The sequence optimization algorithm used comprised generations of diversification and selection, with diversification consisting of recombining all mutations identified from the pool of ancestrally reconstructed sequences, which were embedded in both ESM-1b and LASR (as described above). In silico commenced with the wild-type PTE variant, for which all identified mutations were made upon, and then the 250 sequences with the highest predicted catalytic efficiency were selected passed to the next generation of diversification. Using conventional hardware without background processes running, an overall >20-fold reduction in the wall-time required for the full generational inference on a per sequence basis between LASR (~30 seconds per 20000 sequences) and ESM-1b (~10 minutes per 20000 sequences) was observed (FIG. 12). Accordingly, considering only the wall-time required for sequence embedding, the methods and systems for using LASR as described herein demonstrate an approximately 60-fold speedup over ESM-1b. Over 15 generations of in silico evolution, the activity of variants from LASR-based in silico evolution converged within 5 generations of evolution (1 min 2 seconds).

Accordingly, in this example, having established that LASR representations exceed state-of-the-art performance on supervised tasks for PTE, the relationship between the structure of each model's representation space and predictive performance was determined. Since representations that embed sequences in such a way as to make the fitness function smooth require a less complex transformation to the fitness domain than representations that yield rugged representation spaces, a spectral graph approach was used to assess how rugged/smooth the observed fitness is under each model's representation embedding for the PTE dataset. A normalized Dirichlet energy was used as a smoothness metric on k-nearest neighbor (KNN) graph of the embedded PTE dataset (where k=sqrt (number of sequences)), where nodes represent embedded sequence coordinates and edges connect the k-closest neighbors between each embedded sequence. In general, Dirichlet energy is a measure of how non-smooth a function is. In this example, the local Dirichlet energy is the squared difference in observed fitnesses between adjacent nodes in the KNN graph, while in KNNs where nearby nodes map to similar numerical fitness values, the Dirichlet energy is low, and the embedded fitness landscape can be said to be smooth. The OHE space serves as a control representation of the discrete sequence domain. The fitness function on the KNN graph obtained in the OHE space was expected to have a larger Dirichlet energy than on KNN graphs of the machine-learned representations, implying that PLMs implicitly smooth the discrete sequence domain with respect to the fitnesses.

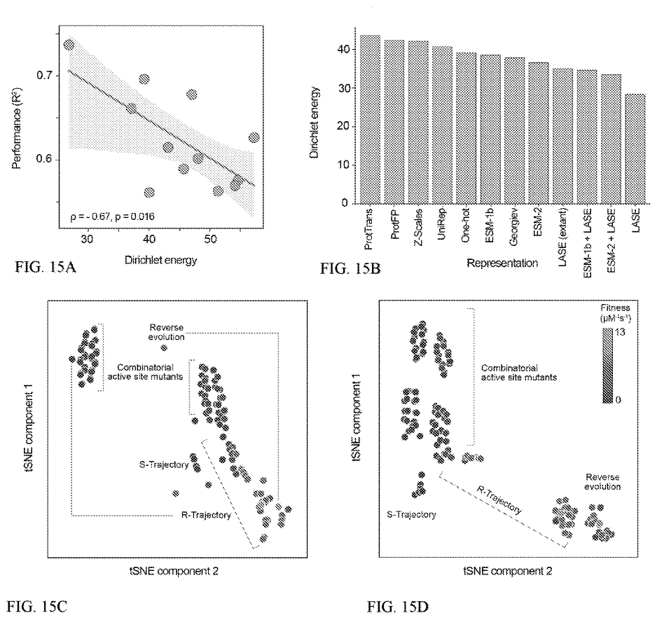

Across all tested representations (including OHE), a negative relationship between the normalized Dirichlet energy of representations' embedded KNN graphs and the random forest regression model's predictive performance (Pearson's R2 correlation coefficient) (R2=0.82, P=0.002) was found (FIG. 15A), indicating that smooth representation spaces (with respect to the fitness) generally produce more predictive (informative) representations in the PTE dataset. Accordingly, LASR yielded the smoothest embedded KNN-graph (FIG. 15B), consistent with performance on arylesterase activity predictions on the PTE test set (Table 2, below).

| Representation | Interpolation performance |
|---|---|
| One-hot | 0.60 |
| Z-Scales | 0.57 |
| ProtFP | 0.58 |
| Georgiev | 0.59 |
| UniRep | 0.56 |
| ProtTrans | 0.63 |
| ESM-1b | 0.69 |
| ESM-2 | 0.62 |
| LASR | 0.76 |
| LASR (extant only control) | 0.56 |

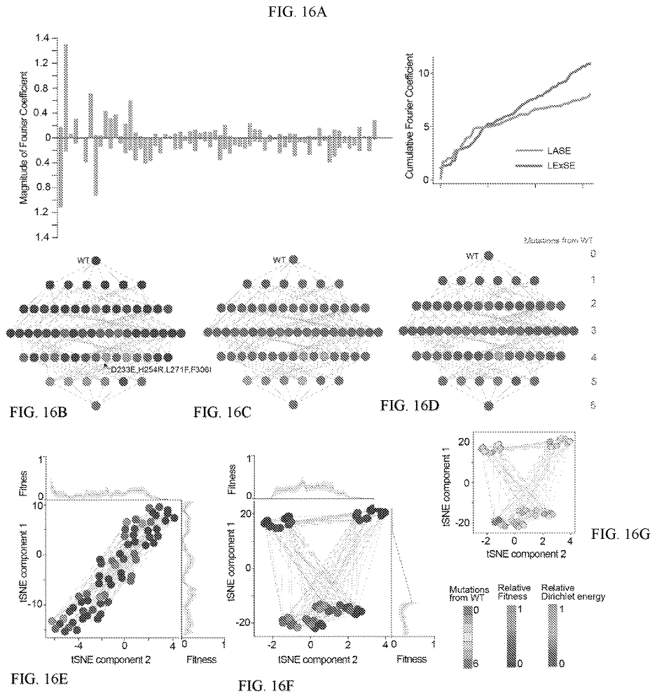

When the representations are projected onto a 2-dimensional t-distributed stochastic neighbor embedding (tSNE) basis for visualization, ESM-2, as shown in FIG. 15D, projections resemble the underlying Hamming graph structure. Notably, the S-Trajectory variants (containing PTE variants with high arylesterase activity) are projected separately from R-Trajectory variants (which also contain variants with high arylesterase activity). In contrast, projections of the LASR embedding space, as shown in FIG. 15C, group the functional variants as neighbors, despite the functional variants having disparate evolutionary history. In turn, a clear functional gradient along both tSNE components, as shown in FIG. 16F, was found, which is seen clearly on analysis of the combinatorial mutants alone which hold a highly structured Hamming space. Variants in the Hamming space are arranged with a functional gradient that is not the result of the underlying data structure, as shown in FIG. 16G, or the general trend for arylesterase activity to improve as more combinatorial mutations are fixed, as shown in FIG. 16B Overall, for this example, the results together demonstrate that LASR implicitly smooths fitness maps during MLM training without explicit regularization, which significantly improves predictive performance over state of the art.

For this example, node-wise Dirichlet energy was used as a measure of epistasis, where epistasis is a measure of how “out of place” (e.g., whether the activity of the sequence is unexpected or unpredictable) a node is in a representation space, with respect to activity. In this example, both a graph signal processing and a graph spectral decomposition approach were used. First, the Dirichlet energy was computed of each node's immediate neighborhood from the KNN graph. The Dirichlet energy was computed in order to determine the fitness of a node, as, if the energy of a node-wise subgraph is high, the fitness of that node deviates in an unpredictable way from its immediate neighbors and the local representation space is rugged with respect to fitness. Additionally, a graph spectral decomposition was performed over the graph to show LASR produces a smoother (and thus more informative) representation space (FIG. 16F), than an equivalent model trained on only extant sequence data as shown in FIG. 16E. Accordingly, where the node-wise Dirichlet energy is a metric of how “strained” a node is given its context in the graph, spectral decomposition provides insight on the contributions that low frequency eigenmodes from the graph topology make towards the observed fitness landscape. When the fitness landscape over a graph is smooth, the fitness landscape is decomposed into low frequency (i.e., simple) eigenmodes with greater magnitudes than high frequency (e.g., complex) eigenmodes. Indeed, when sequences are represented in the OHE domain and the graph is the fully-connected hamming graph, spectral decomposition provides the magnitudes that different orders of epistasis contribute to the observed fitness landscape. If the combinatorial PTE fitness map is smoother as a KNN graph in an embedded space than the equivalent in the OHE space, the embedding model's learned representation space reduces non-linearity imposed by epistasis.

The topological features of LASR and the equivalent extant only control model (LEx; LASR (extant only) embedded PTE fitness maps were compared to assess whether ancestrally reconstructed sequences are fundamentally more informative in learning epistasis than their extant counterparts. Lower (and hence less ‘strained’) local dirichlet energies were systematically observed in the LASR representation space (FIG. 16D) than the LEx representation space (FIG. 16C). Interestingly, while the LASR KNN graph is less strained than other representations, local energies in each graph follow the same general node-wise structure, indicating that the sequences most confounded by epistasis are common between representations and embedding in a representation model only dampens complexity in the fitness map, rather than drastically altering it.

### Example System for Generating Result Sequences

FIG. 2 depicts an example system 200 for generating a plurality of result sequences 230 (e.g., biological sequences) based on one or more test sequence(s) 210. In this depicted example, system 100 comprises a server 110 and a computing device 240. In some embodiments, the system 100 does not include the computing device 240. In some embodiments, system 200 may also include computing device 120 of FIG. 1.

In this depicted embodiment, server 110 further includes ancestral reconstruction component 112, evolution component 114, and refinement component 116.

In this depicted example, ancestral reconstruction component 112 is configured to receive test sequence(s) 210 from computing device 240. Ancestral reconstruction component 112 may further be configured to utilize the test sequence(s) 210 to generate a plurality of reconstructed sequences based on the test sequence(s) 210. The plurality of reconstructed sequences may be generated by one or more techniques for ancestral reconstruction. In some embodiments, the one or more techniques includes using a machine learning model (e.g., the trained reconstruction machine learning model of FIG. 1) to output the plurality of reconstructed sequences with the test sequence(s) 210 as input. In other embodiments, the one or more techniques includes a statistical algorithm such as a maximum likelihood ancestral sequence reconstruction, empirical Bayesian ancestral sequence reconstruction, hierarchical Bayesian ancestral sequence reconstruction and/or maximum parsimony.

Further, the ancestral reconstruction component 112 may be configured to provide the plurality of reconstructed sequences to evolution component 114. Evolution component 114 may be configured to generate a plurality of evolved sequences based on the plurality of reconstructed sequences. In this depicted embodiment, evolution component 114 utilizes machine learning model 118 to generate the plurality of evolved sequences based on the plurality of reconstructed sequences. The machine learning model 118 may generate the plurality of evolved sequences by modifying one or more of the plurality of reconstructed sequences (e.g., by adding or removing at least one amino acid from the one or more sequences and/or substituting at least one amino acid from the one or more sequences). The plurality of evolved sequences may include both the evolved sequences generated by machine learning model 118 based on the modifications and the plurality of reconstructed sequences. In some embodiments, the plurality of evolved sequences may only include the evolved sequences generated by machine learning model 118.

Further, the evolution component 114 may be configured to provide the plurality of evolved sequences to refinement component 116. In this depicted embodiment, refinement component also receives user input 220 from computing device 240. Further, in this depicted embodiment, computing device 240 further includes UI component 222, wherein the UI component 122 is configured to receive user input 220 through a user interface. The user input 220 may include one or more of a percent identity threshold, the robustness of one or more sequences, the stability of one or more sequences, an activity of one or more sequences, an expression of one or more sequences, or combinations thereof. The user input 220 may also include one or more sequences related to the percent identity threshold. In this depicted embodiment, refinement component 116 is configured to generate a plurality of result sequences based on the plurality of evolved sequences and the user input. In some embodiments, the refinement component 116 is configured to generate the plurality of result sequences based on only the plurality of evolved sequences. In some embodiments, generating the plurality of result sequences includes using a machine learning model (e.g., the trained refinement machine learning model described with respect to FIG. 1).

Server 110 may be configured to provide the plurality of result sequences to the computing device 240 after generating the plurality of result sequences. In some embodiments, the server 110 is configured to store one or more of the test sequence(s) 210, the user input 220, the plurality of reconstructed sequences, the plurality of evolved sequences, and the plurality of result sequences in a database. In some embodiments, the database may be included in the server 110. In some embodiments, the database may be stored in a network. In some embodiments, test sequence(s) 210 and/or one or more sequences of plurality of result sequences 230 may be displayed on UI component 122.

### Example Generation of Plurality of Reconstructed Sequences

FIG. 3 depicts an example process for generating a plurality of reconstructed sequences. In this depicted example, ancestral reconstructor 310 receives a test sequence 312 and generates a plurality of sequences 318. In some embodiments, ancestral reconstructor 310 may be included on a server (e.g., in ancestral reconstruction component 112 of server 110 of FIGS. 1-2). In some embodiments, ancestral reconstructor 310 may be a machine learning model (e.g., the reconstruction machine learning model as described with respect to FIG. 1). In other embodiments, ancestral reconstructor 310 may generate the plurality of reconstructed sequences as described with respect to FIG. 1. In some embodiments, ancestral reconstructor 310 may generate the plurality of sequences 318 by generating a phylogenetic tree.

In this depicted embodiment, ancestral reconstructor 310 receives test sequence 312, where test sequence 312 has an exemplary amino acid sequence “ABCDEF”. While sequence ABCDEF and individual amino acids A, B, C, D, E, and F are depicted, this sequence and individual amino acids are exemplary and other sequences and amino acids may be used.

Ancestral reconstructor 310 proceeds to generate plurality of reconstructed sequences 318 by masking portions of test sequence 318 and generating sequences based on those masked portions. In this depicted example, ancestral reconstructor 310 masks portion 314a (covering amino acids “BCDE”) in a first iteration of phylogenetic inference as well as 314b (covering amino acid “A”) and 314c (covering amino acids “EF”) in a second iteration of phylogenetic inference. In some embodiments, portions of any length of amino acid sequence can be masked (e.g., masking at least one amino acid (e.g., portion 314b) and up to all amino acids). In some embodiments, more than one portion of the amino acid sequence may be masked (e.g., portions 314b and 314c being masked in the same iteration). Ancestral reconstructor 310 then proceeds to generate a plurality of reconstructed sequences based on masked portion 314a as well as 314b and 314c.

In some embodiments, the ancestral reconstructor 310 may be a machine learning model. In some embodiments, ancestral reconstructor 310 may be a generative adversarial network machine learning model. In some embodiments, the ancestral reconstructor 310 may use a kernel mechanism. In some embodiments, the ancestral reconstructor 310 may be a convolutional neural network. In some embodiments, the ancestral reconstructor 310 uses an attention or a multi-head attention mechanism. In some embodiments, the ancestral reconstructor 310 is a transformer.

For example, in this depicted embodiment, based on masked portion 314a, ancestral reconstructor 310 generates plurality of sequences 316a. Plurality of sequences 316a is generated based on possible sequences of amino acid that could be substituted for the masked portion. In some embodiments, the sequences substituted for the masked portion may include new amino acids in the position of previous amino acids, amino acids that are added to the sequence, or amino acids that have been removed from the sequence. For example, test sequence 312, with a sequence of ABCDEF, having a masked portion 314a (“BCDE”), may lead to sequences AEDBCF, ABDECF, ADBCEF, ABBCEF, and ABCAEF where one of the amino acids in the masked portion 314a is changed from “BCDE”.

Similarly, in this depicted embodiment, based on masked portions 314b and 314c, ancestral reconstructor 310 generates plurality of sequences 316b. For example, test sequence 312, with a sequence of ABCDEF, having masked portions 314b and 314c, may lead to sequences FBCDEA, ABCDFE, BBCDFE, EBCDEA, and ABCDFF, where at least one of the amino acids in the masked portions 314b and 314c is changed.

In this depicted example, generated ancestrally reconstructed sequences 316a and 316b are included with test sequence 312 to create plurality of reconstructed sequences 318 to be used in generating result sequences. While only five masked sequences are depicted as being generated based on each of the masked portions, the five masked sequences are exemplary, and more or fewer sequences may be generated based on the masked sequences. Further, while a certain number of sequences are shown in plurality of reconstructed sequences 318, this number is exemplary, and more or fewer sequences may be generated. Additionally, if ancestral reconstructor 310 is a machine learning model, the ancestral reconstructor 310 may receive feedback to further train the machine learning model. In some embodiments, the feedback may include a refined plurality of reconstructed sequences (e.g., refined plurality of reconstructed sequences 150 of FIG. 1) that includes one or more extra reconstructed sequences and/or does not include certain sequences that were ancestrally reconstructed by ancestral reconstructor 130 for further training the machine learning model.

In other embodiments, ancestral reconstructor 310 may not generate the plurality of reconstructed sequences 318 by masking portions of test sequence 318, and may instead generate the plurality of reconstructed sequences 318 by using another method. For example, other methods may include “next token prediction”. When using next token prediction, one or more initial portions of a test sequence (e.g., test sequence 312) may be provided to ancestral reconstructor 310. Ancestral reconstructor 310 may then determine at least one ending portion for each of the one or more initial portions of the test sequence. For example, using a test sequence of “ABCDEF”, initial portions “AB”, “ABC”, and “ABCD” of the test sequence may be provided to the ancestral reconstructor 310 as input. Ancestral reconstructor 310 may then generate one or more ending portions for each initial portion, and may output the ending portions and/or the sequences created by combining initial portions with corresponding ending portions. For example, using the initial portion “AB”, the ancestral reconstructor 310 may generate ending portions, “CDEE”, “CDFE”, “DCEC”, or additional ending portions for the initial portion “AB”. As part of the same example, using the initial portion “ABC”, the ancestral reconstructor 310 may generate ending portions, “DFE”, “BDE”, “AFC”, or additional ending portion for the initial portion “ABC”. As part of the same example, using the initial portion “ABCD”, the ancestral reconstructor 310 may generate ending portions, “DE”, “CE”, “FE”, “FED”, or additional ending portions for the initial portion “ABCD”. The ancestral reconstructor 310 could then output the generated ending portions and/or sequences created by combining the initial portions with corresponding ending portions (e.g., “ABCDEE”, “ABCDFE”, “ABDCEC”, “ABCDFE”, “ABCBDE”, “ABCAFC”, “ABCDDE”, “ABCDCE”, and “ABCDFED”). If ancestral reconstructor 310 is a machine learning model, the ancestral reconstructor 310 may receive feedback to further train the machine learning model. In some embodiments, the feedback may include a refined plurality of reconstructed sequences (e.g., refined plurality of reconstructed sequences 150 of FIG. 1) that includes one or more extra reconstructed sequences and/or does not include certain sequences that were ancestrally reconstructed by ancestral reconstructor 130 for further training the machine learning model.

### Example Generation of Plurality of Evolved Sequences

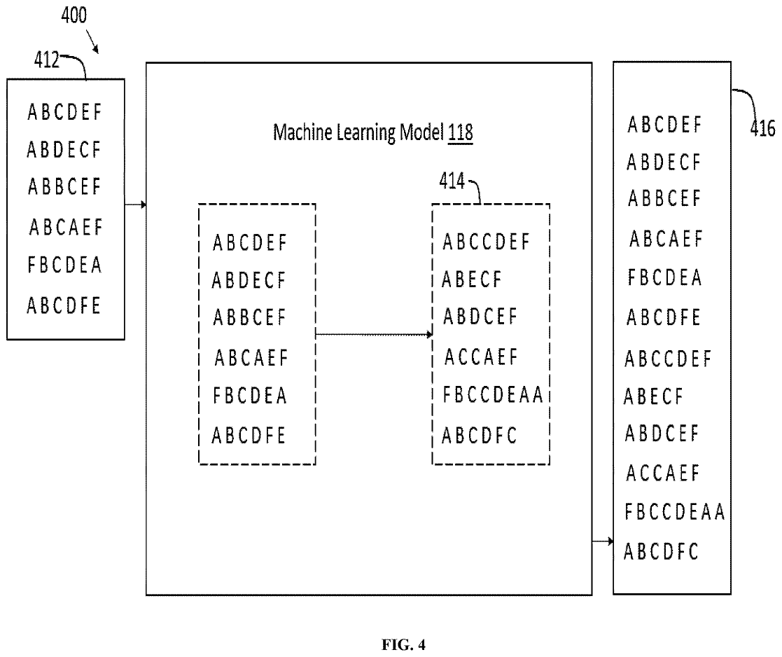

FIG. 4 depicts an example process for generating an evolved plurality of sequences (e.g., biological sequences; evolved plurality of sequences 160 of FIG. 1) with machine learning model 118. In some embodiments, machine learning model 118 may be included on a server (e.g, in evolution component 114 of server 110 of FIG. 1).

In this depicted embodiment, the machine learning model 118 receives a plurality of reconstructed sequences 412. In this depicted embodiment, plurality of reconstructed sequences 412 has been refined from a larger set of reconstructed sequences based on one or more refinement processes. In some embodiments, the one or more refinement processes may include removing one or more sequences that are undesired due to one or more indications associated with the sequences, such as robustness, stability, activity, or length of the sequence. In some embodiments, the larger set of reconstructed sequences is an unrefined plurality of reconstructed sequences (e.g., plurality of reconstructed sequences 140 of FIG. 1 or plurality of reconstructed sequences 318 of FIG. 3). In some embodiments, the plurality of reconstructed sequences 412 is an unrefined plurality of reconstructed sequences (e.g., plurality of refined reconstructed sequences 150 of FIG. 1).

In this depicted embodiment, machine learning model 118 proceeds to evolve at least one of the plurality of reconstructed sequences 412. Evolving a reconstructed sequence may include making one or more modifications to the sequences, such as removing or adding one or more amino acids from a sequence of the plurality if reconstructed sequences, or substituting one or more amino acids from a sequence of the plurality of reconstructed sequences. For example, in this depicted embodiment, each of the plurality of reconstructed sequences 412 has been evolved in at least one way by machine learning model 118 to create evolved sequences 414 (e.g., the sequence ABDEF is evolved into the sequence “ABCDEF” is evolved by inserting a “C” amino acid into the third position, sequence ABCEF is evolved by deleting a “D” amino acid from the sequence ABCDEF, the sequence ABBDEF is evolved by substituting a “C” amino acid with a “D” amino acid in the sequence ABCDEF, etc.). While each sequence of the received plurality of reconstructed sequences is evolved in such a way to create one corresponding evolved sequence, this is exemplary, and each sequence of the received plurality of reconstructed sequences may be evolved in multiple ways in one reconstructed sequence as well as evolved to create more than one corresponding evolved sequence.

In this depicted embodiment, machine learning model 118 then outputs plurality of evolved sequences 416. In this depicted embodiment, plurality of evolved sequences 416 includes both the received plurality of reconstructed sequences 412 and the evolved sequences 414 generated by the machine learning model 118. In some embodiments, the plurality of evolved sequences 416 only includes the generated evolved sequences by the machine learning model 118. Machine learning model 118 may then provide the plurality of evolved sequences for use in generating a plurality of result sequences.

In some embodiments, machine learning model 118 may receive feedback regarding the plurality of evolved sequences 416 for further training of machine learning model 118. In some embodiments, the feedback includes a refined plurality of evolved sequences, which may have one or more sequences of the plurality of evolved sequences 416 removed and/or may have one or more new evolved sequences added.

### Example Generation of Plurality of Result Sequences

FIG. 5 depicts an example process for generating a plurality of result sequences (e.g, plurality of result sequences 180 of FIG. 1) with refinery 510. In some embodiments, refinery 510 may be included on a server (e.g., in refinement component 116 of server 110 of FIG. 1).

In this depicted embodiment, refinery 510 receives plurality of evolved sequences 512. In some embodiments, the plurality of evolved sequences may be generated by a machine learning model (e.g., generated by machine learning model 118 as described with respect to FIG. 4).

Refinery 510 may refine a plurality of evolved sequences 512 by removing one or more sequences of the plurality of evolved sequences 512 based on one or more indications. The one or more indications may include a respective robustness of each evolved sequence in plurality of evolved sequences 512, a respective stability of each evolved sequence in plurality of evolved sequences 512, a respective activity of each evolved sequence in plurality of evolved sequences 512, a respective expression of each evolved sequence in plurality of evolved sequences 512, a redundancy threshold with respect to chosen sequence such as a test sequence or a model sequence (e.g., test sequence(s) 210 of FIG. 2 or an indicated sequence included in user input 220), a threshold sequence length, or combinations thereof. Thus, refinery 510 may remove sequences from the evolved plurality of sequences if the sequences do not meet the defined value of the indication, e.g., the robustness, stability, activity, and/or expression of a sequence does not meet or exceed the robustness, stability, activity, and/or expression as defined by the indication, the sequence does not reach a defined percent identity as compared to a chosen sequence, and/or the sequence is longer or shorter than a length defined by the indication. The indications and/or values for the indications may be received by the refinery. The indications and/or values for the indications may also be a part of received user input. While certain indications are defined above, these indications are exemplary and other indications may be used.

In this depicted example, refinery 510 removes sequences from the plurality of evolved sequences 512 based on indication 514 and indication 516. For example, indication 514 may be defined as a threshold robustness and indication 516 may be defined as a threshold length. Refinery 510 applies the indications to the sequences of the plurality of evolved sequences 512. Thus, refinery 510 then removes all sequences that have a robustness lower than the threshold robustness or a length that is longer than the threshold length, and output the remaining sequences as plurality of result sequences 518.

Thus, refinery 510 is configured to receive a plurality of evolved sequences and apply indications to the plurality of evolved sequences in order to generate a plurality of result sequences, and later provide the plurality of result sequences.

### Example Method for Training a Machine Learning Model

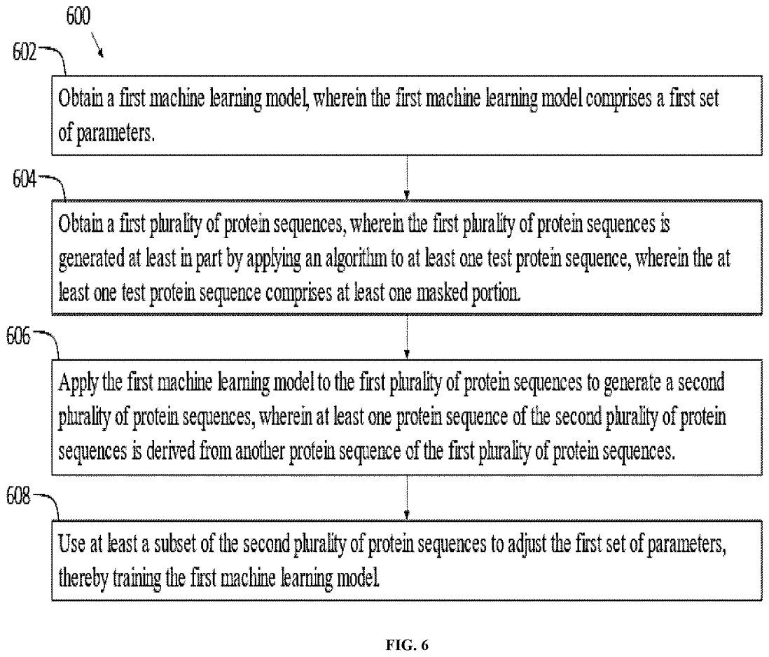

FIG. 6 depicts an example method for training a first machine learning model (e.g., machine learning model 118). In some embodiments, the first machine learning model may be utilized by a processing device (e.g., server 110 of FIGS. 1-2) that may be in communication with another processing device (e.g., computing device 120 of FIG. 1).

The method begins at step 602 by obtaining the first machine learning model, which includes a first set of parameters. The parameters are related to evolving one or more biological sequences (e.g., amino acid/protein sequences or nucleotide sequences) received by the model.

At step 604, a first plurality of sequences is obtained (e.g., plurality of reconstructed sequences 140 or plurality of refined reconstructed sequences 150 of FIG. 1), where the first plurality of sequences is generated at least in part by applying an algorithm to at least one test sequence (e.g., ancestral reconstruction component 112 generating plurality of reconstructed sequences 140 which may be later refined to refined plurality of reconstructed sequences 150 based on test sequence(s) 130 as described with respect to FIG. 1). In some embodiments, the algorithm may be a second machine learning model (e.g., the reconstruction machine learning model as described with respect to FIG. 1). The second machine learning model may be an ASR model. The second machine learning model may be trained based on feedback, such as a refined plurality of reconstructed sequences, received by the model. In some embodiments, the algorithm may be a statistical algorithm, such as maximum likelihood ancestral sequence reconstruction, empirical Bayesian ancestral sequence reconstruction, hierarchical Bayesian ancestral sequence reconstruction, or maximum parsimony ancestral sequence reconstruction. The first plurality of sequences may be generated during or after phylogenetic inference (e.g., by ancestral reconstruction component 112), where the phylogenetic inference was performed using the statistical algorithm. In some embodiments, the first plurality of sequences may include no more than 500 sequences, but in other embodiments may include thousands of sequences (e.g., ten thousand sequences).

At step 606, the first machine learning model is applied to the first plurality of sequences to generate a second plurality of sequences (e.g., plurality of evolved sequences 160 of FIG. 1), where at least one sequence of the second plurality of sequences is derived (e.g., evolved by adding, removing, and/or substituting one or more amino acids) from another sequence of the first plurality of sequences. In some embodiments, the first machine learning model is a Learned Ancestral Sequence Reconstruction (“LASR”) model initialized with random parameters. In some embodiments, the first machine learning model may be a model initialized from parameters trained previously on protein and/or nucleotide sequences, such as a UniRep model, a transformer model, a LSTM model, or RNN model. In some embodiments, the first machine learning model is a transformer model. In some embodiments, the first machine learning model is a transformer model. In some embodiments, the first machine learning model is CNN. In some embodiments, the first machine learning model may generate the second plurality of sequences based at least in part on a first list containing a distribution of filtered sequence lengths of the second plurality of sequences per percent identity redundancy based on a redundancy threshold and/or a second list containing a number of sequences of the second plurality of sequences per percent identity based on the redundancy threshold. In some embodiments, the second plurality of sequences may be provided to another processing device (e.g., computing device 120) in order to receive feedback from the processing device. In some embodiments, the feedback may include a subset of the second plurality of sequences (e.g., refined plurality of evolved sequences 170 of FIG. 1). In some embodiments, the second plurality of sequences may further include a plurality of accession numbers corresponding to the sequences of the second plurality of sequences a plurality of accession numbers associated with experimental validation, wherein the plurality of accession numbers corresponds to a first subset of the second plurality of sequences, and/or a plurality of accession numbers associated with solved crystal structures, wherein the plurality of accession numbers corresponds to a second subset of the second plurality of sequences.

At step 608, the subset of the second plurality of sequences may be used to adjust the first set of parameters, thus training the machine learning model.

In some embodiments, the subset of the second plurality of sequences may be used to generate a third plurality of sequences (e.g., plurality of result sequences 180 of FIG. 1). In some embodiments, a third machine learning model (e.g., the refinement machine learning model as described with respect to FIG. 1) may be used to generate the third plurality of sequences. The third plurality of sequences may be generated by removing or adding one or more sequences or portions of sequences from the subset of the second plurality of sequences based on one or more indications. The indications may be retrieved from a database or may be received as user input. The indications may include one or more of a percent identity threshold to one or more selected sequences, the robustness of one or more sequences, the stability of one or more sequences, an activity of one or more sequences, an expression of one or more sequences, or combinations thereof. In some embodiments, the percent identity threshold may be about 50%, about 55%, about 60%, about 65%, about 70%, about 75%, about 80%, about 85%, about 90%, about 95%, about 96%, about 97%, about 98%, or about 99%. In some embodiments, the one or more selected sequences may include test sequence(s) 130.

In some embodiments, the one or more sequences of the first, second, and/or third plurality of sequences may be stored in a database. In some embodiments, the one or more sequences of the first, second, and/or plurality of sequences may be displayed.

### Example Method for Generating Result Sequences

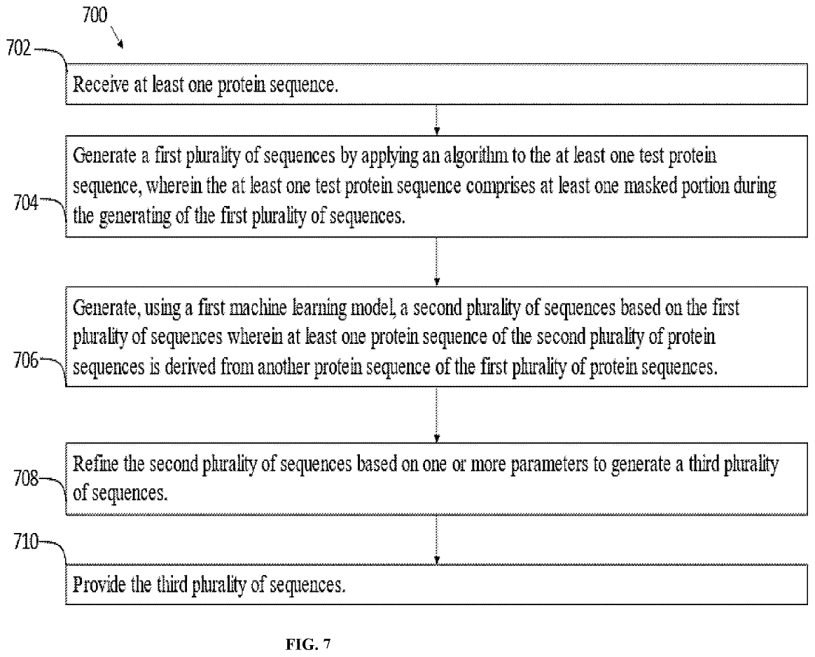

FIG. 7 depicts an example method for generating one or more pluralities of biological sequences (e.g., amino acid/protein sequences or nucleotide sequences). In some embodiments, a processing device (e.g., server 110 of FIGS. 1-2) may be used to generate the one or more pluralities of sequences (e.g., plurality of reconstructed sequences 140), and may be in communication with another processing device (e.g., computing device 240 of FIG. 2).

The method begins at step 702 by receiving at least one sequence (e.g., test sequence 210 of FIG. 2).

At step 704, a first plurality of sequences is generated by applying an algorithm to the at least one test sequence, wherein the at least one test sequence comprises at least one masked portion during the generating of the first plurality of sequences. In some embodiments, the algorithm may be a machine learning model (e.g., the reconstruction machine learning model as described with respect to FIG. 1). In some embodiments, the algorithm may be a statistical algorithm, such as maximum likelihood ancestral sequence reconstruction, empirical Bayesian ancestral sequence reconstruction, hierarchical Bayesian ancestral sequence reconstruction, or maximum parsimony ancestral sequence reconstruction. The first plurality of sequences may be generated during or after phylogenetic inference (e.g., by ancestral reconstruction component 112 of FIG. 1), where the phylogenetic inference was performed using the statistical algorithm.

At step 706, a second plurality of sequences is generated by a first machine learning model (e.g., machine learning model 118 of FIGS. 1, 2, and 4) based on the first plurality of sequences, whereat least one sequence of the second plurality of sequences is derived from another sequence of the second plurality of sequences is derived from another sequence of the first plurality of sequences. The second plurality of sequences may be derived by evolving another sequence (e.g., by adding, removing, and/or substituting one or more amino acids).

At step 708, a third plurality of sequences (e.g., plurality of result sequences 230) is generated by refining the second plurality of sequences based on one or more indications (e.g., indication 514 and indication 516 of FIG. 5). The one or more indications may include a respective robustness of each sequence, a respective stability of each sequence, a respective activity of each sequence, a respective expression of each sequence, a redundancy threshold with respect to chosen sequence such as a test sequence or a model sequence, a threshold sequence length, or combinations thereof. Thus, sequences that do not meet the defined value of the indication may be removed, e.g., the robustness, stability, activity, and/or expression of a sequence does not meet or exceed the robustness, stability, activity, and/or expression as defined by the indication, the sequence does not reach a defined percent identity as compared to a chosen sequence, and/or the sequence is longer or shorter than a length defined by the indication. The indications and/or values for the indications may be received as user input (e.g., user input 220 of FIG. 2).

At step 710, the third plurality of sequences may be provided to another processing device (e.g., computing device 120 of FIGS. 1-2).

### Computer Systems

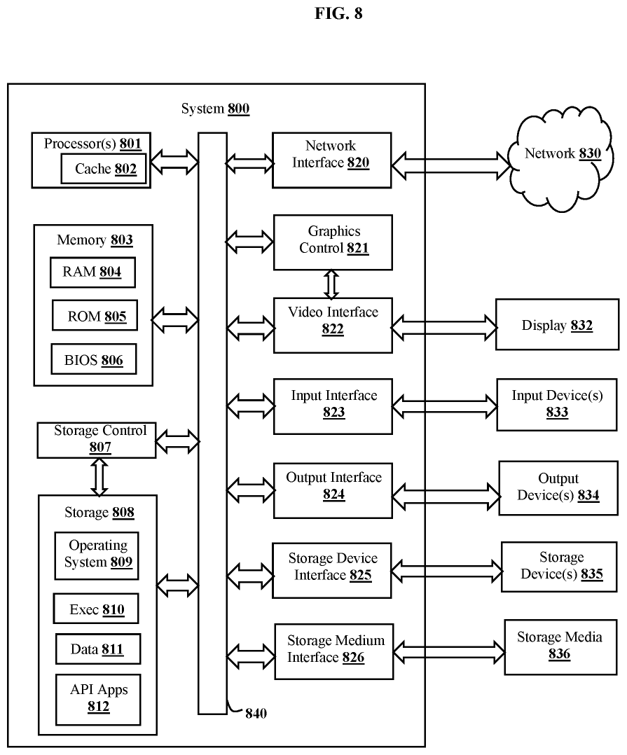

The present disclosure provides computer systems that are programmed to implement methods of the disclosure. FIG. 8 shows a computer system 801 that is programmed or otherwise configured to generate (e.g., predict) various pluralities of sequences and output those pluralities of sequences. The computer system 801 may be configured to regulate various aspects generating and outputting the various pluralities of sequences. The computer system 801 may be an electronic device of a user or a computer system that is remotely located with respect to the electronic device. The electronic device can be a mobile electronic device. In some embodiments, the statistical algorithms and/or machine learning models described above (e.g., machine learning model 118 of FIGS. 1-2) can be utilized on the same electronic device.

The computer system 801 includes a central processing unit (CPU, also “processor” and “computer processor” herein) 805, which may be a single core or multi core processor, or a plurality of processors for parallel processing. The computer system 801 also includes memory or memory location 810 (e.g., random-access memory, read-only memory, flash memory), electronic storage unit 815 (e.g., hard disk), communication interface 820 (e.g., network adapter) for communicating with one or more other systems, and peripheral devices 825, such as cache, other memory, data storage, graphical processing units (GPU), tensor processing units (TPU), and/or electronic display adapters. The memory 810, storage unit 815, interface 820 and peripheral devices 825 are in communication with the CPU 805 through a communication bus (solid lines), such as a motherboard. The storage unit 815 can be a data storage unit (or data repository) for storing data. The computer 801 peripheral devices 825 (for example, GPU) and CPU 805 may share unified system memory 810. The computer 801 CPU 805, memory 810 and peripheral devices 825 (for example, GPU) may be separate components on the same silicon chip. The computer system 801 may be configured to be operatively coupled to a computer network (“network”) 830 with the aid of the communication interface 820. The network 830 can be the Internet, an internet and/or extranet, or an intranet and/or extranet that is in communication with the Internet. The network 830 in some cases is a telecommunication and/or data network. The network 830 may be configured to include one or more computer servers, which can enable distributed computing, such as cloud computing. The network 830, in some cases with the aid of the computer system 801, can implement a peer-to-peer network, which may enable devices coupled to the computer system 801 to behave as a client or a server.

The CPU 805 may be configured to execute a sequence of machine-readable instructions, which can be embodied in a program or software. The instructions may be stored in a memory location, such as the memory 810. The instructions may be directed to the CPU 805, which may subsequently program or otherwise configure the CPU 805 to implement methods of the present disclosure. Examples of operations performed by the CPU 805 can include fetch, decode, execute, and writeback.

The CPU 805 may be configured to be part of a circuit, such as an integrated circuit. One or more other components of the system 801 may be included in the circuit. In some cases, the circuit is an application specific integrated circuit (ASIC).

The storage unit 815 may be configured to store files, such as drivers, libraries and saved programs. The storage unit 815 can store user data, e.g., user preferences and user programs. The computer system 801 in some cases can include one or more additional data storage units that are external to the computer system 801, such as located on a remote server that is in communication with the computer system 801 through an intranet or the Internet.

The computer system 801 may be configured to communicate with one or more remote computer systems through the network 830. For instance, the computer system 801 may be configured to communicate with a remote computer system of a user (e.g., computing device 104 of FIG. 1). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user may access the computer system 801 via the network 830.

Methods as described herein can be implemented by way of machine (e.g., computer processor) executable code stored on an electronic storage location of the computer system 801, such as, for example, on the memory 810 or electronic storage unit 815. The machine executable or machine readable code can be provided in the form of software. During use, the code can be executed by the processor 805. In some cases, the code can be retrieved from the storage unit 815 and stored on the memory 810 for ready access by the processor 805. In some situations, the electronic storage unit 815 can be precluded, and machine-executable instructions are stored on memory 810.

The code may be pre-compiled and configured for use with a machine having a processer adapted to execute the code, or can be compiled during runtime. The code may be supplied in a programming language that can be selected to enable the code to execute in a pre-compiled or as-compiled fashion.

Aspects of the systems and methods provided herein, such as the computer system 801, may be embodied in programming. Various aspects of the technology may be thought of as “products” or “articles of manufacture” typically in the form of machine (or processor) executable code and/or associated data that is carried on or embodied in a type of machine readable medium. Machine-executable code may be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 801 can include or be in communication with an electronic display 835 that comprises a user interface (UI) 840 for providing, for example, user input associated with or use for generating various pluralities of sequences. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 805. The algorithm can, for example, allow the system 801 to generate various pluralities of sequences.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, XML, and document oriented database systems. In further embodiments, suitable relational database systems include, by way of non-limiting examples, Microsoft® SQL Server, mySQL™, and Oracle®. A web application, in various embodiments, may be written in one or more versions of one or more languages. A web application may be written in one or more markup languages, presentation definition languages, client-side scripting languages, server-side coding languages, database query languages, or combinations thereof. In some embodiments, a web application may be written to some extent in a markup language such as Hypertext Markup Language (HTML), Extensible Hypertext Markup Language (XHTML), or extensible Markup Language (XML). In some embodiments, a web application may be written to some extent in a presentation definition language such as Cascading Style Sheets (CSS). In some embodiments, a web application is written to some extent in a client-side scripting language such as Asynchronous Javascript and XML (AJAX), Flash® ActionScript, JavaScript, or Silverlight®. In some embodiments, a web application may be written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application may include a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

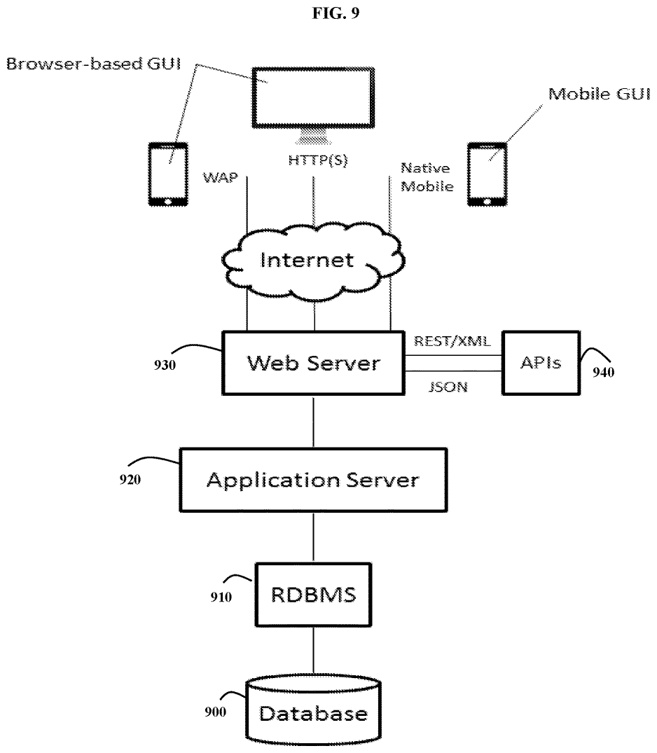

Referring to FIG. 9, in a particular embodiment, an application provision system comprises one or more databases 900 accessed by a relational database management system (RDBMS) 910. Suitable RDBMSs include Firebird, MySQL, PostgreSQL, SQLite, Oracle Database, Microsoft SQL Server, IBM DB2, IBM Informix, SAP Sybase, Teradata, and the like. In this embodiment, the application provision system further comprises one or more application severs 920 (such as Java servers, .NET servers, PHP servers, and the like) and one or more web servers 930 (such as Apache, IIS, GWS and the like). The web server(s) optionally expose one or more web services via app application programming interfaces (APIs) 940. Via a network, such as the Internet, the system provides browser-based and/or mobile native user interfaces.

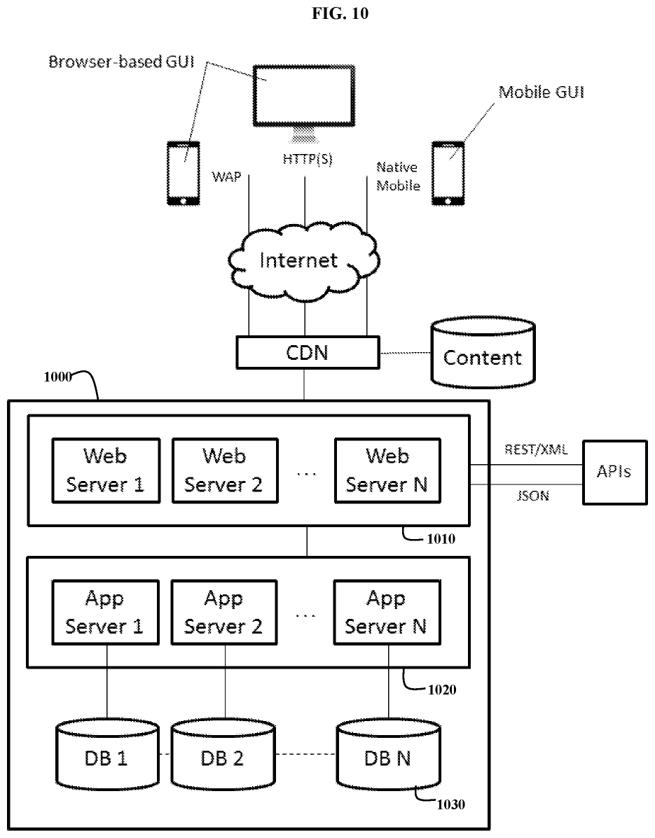

Referring to FIG. 10, in a particular embodiment, an application provision system alternatively has a distributed, cloud-based architecture 1000 and comprises elastically load balanced, auto-scaling web server resources 1010 and application server resources 1020 as well synchronously replicated databases 1030.

### Mobile Application

In some embodiments, a computer program may include a mobile application provided to a mobile computing device. In some embodiments, the mobile application is provided to a mobile computing device at the time it is manufactured. In other embodiments, the mobile application is provided to a mobile computing device via the computer network described herein.

In view of the disclosure provided herein, a mobile application may be created by using various hardware, languages, and development environments. Mobile applications may be written in several languages. Suitable programming languages include, by way of non-limiting examples, C, C++, C#, Objective-C, Java™, JavaScript, Pascal, Object Pascal, Python™, Ruby, VB.NET, WML, and XHTML/HTML with or without CSS, or combinations thereof.

Suitable mobile application development environments may be available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and PhoneGap. Also, mobile device manufacturers distribute software developer kits including by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Several commercial forums may be available for distribution of mobile applications including, by way of non-limiting examples, Apple® App Store, Google® Play, Chrome Web Store, BlackBerry® App World, App Store for Palm devices, App Catalog for webOS, Windows® Marketplace for Mobile, Ovi Store for Nokia® devices, Samsung® Apps, and Nintendo® DSi Shop.

### Non-Transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

### Software Modules

In some embodiments, the platforms, systems, media, and methods disclosed herein include software, server, and/or database modules, or use of the same. In view of the disclosure provided herein, software modules may be created by using various machines, software, and languages. The software modules disclosed herein may be implemented in a multitude of ways. In various embodiments, a software module may comprise a file, a section of code, a programming object, a programming structure, a distributed computing resource, a cloud computing resource, or combinations thereof. In further various embodiments, a software module may comprise a plurality of files, a plurality of sections of code, a plurality of programming objects, a plurality of programming structures, a plurality of distributed computing resources, a plurality of cloud computing resources, or combinations thereof. In various embodiments, the one or more software modules may comprise, by way of non-limiting examples, a web application, a mobile application, a standalone application, and a distributed or cloud computing application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of sequence information, nucleotide information, generated (e.g., predicted) sequences, received sequences, parameters, parameter values, indications, or indication values. In various embodiments, suitable databases include, by way of non-limiting examples, relational databases, non-relational databases, object oriented databases, object databases, entity-relationship model databases, associative databases, XML databases, document oriented databases, and graph databases. Further non-limiting examples include SQL, PostgreSQL, MySQL, Oracle, DB2, Sybase, and MongoDB. In some embodiments, a database is Internet-based. In further embodiments, a database is web-based. In still further embodiments, a database is cloud computing-based. In a particular embodiment, a database is a distributed database. In other embodiments, a database is based on one or more local computer storage devices.

### EXAMPLES

The following illustrative examples are representative of embodiments of systems and methods described herein and are not meant to be limiting in any way.

### Example 1: Generating Reconstructed Sequences

To being, a processing device receives an amino acid sequence of extant PTE. The amino acid sequence of extant PTE was used as a seed sequence to retrieve and collect >10,000 amino acid sequences with an E-value >10e-3 from a private JGI database of >1,000,000,000 metagenomic reads. This dataset was refined to 499 sequences following a hidden Markov model (HMM) against a profile of PTE and highly-homologous sequences, and by manual inspection. Sequences were aligned using the GINSI protocol of MAFFT. Phylogenetic and ancestral sequence reconstruction was performed in concert by maximum likelihood in IQ-TREE. The ASR algorithm employed in IQ-TREE is identical to the empirical Bayesian algorithm employed in the literature standard PAML suite, however is parallelizable and supports parameter-rich sequence evolution models. 100 independent tree searches and reconstructions were performed for the top-performing models: JTT+F+R7, LG+R10. As a comparison, 100 replicates of tree search were also performed using WAG+G4, a general amino acid replacement matrix that was not selected by any of IQ-TREE's information criteria. This yielded a dataset of ~150,000 diverse ancestral sequences from <500 extant PTE-like sequences. Because the sequence reconstruction model did not account for insertion-deletion (indel) events in the evolutionary process, they were probabilistically processed independently. This was done through an equal-rates maximum likelihood model that inferred the posterior probability of an insertion at a given site of an ancestral sequence, given the presence or absence of that insertion in its descendants along the tree topology. Unlike a parsimonious approach that minimizes the number of changes over the tree, the maximum likelihood indel model provides statistical support for a given insertion, which are detected automatically from the alignment. Ancestral sequence datasets were concatenated and redundancy was removed to 100% sequence identity, yielding a final dataset size of ~8000 unique sequences.

### Example 2: Generating Evolved Sequences

The processing device of Example 1 inputs the plurality of 10,000 protein sequences phylogenetically related to the test protein sequence of Example 1 to an LSTM machine learning model configured to evolve protein sequences to create related sequences. The machine learning model evolves a subset of the 10,000 protein sequences by making additions, deletions, and/or substitutions to the subset of the 10,000 protein sequences to generate a plurality of 20,0000 evolved protein sequences. The processing device later receives a subset of the 20,000 evolved protein sequences as feedback and further trains the LSTM machine learning model by adjusting the parameters of the machine learning model.

The LSTM machine learning model was produced using PyTorch and TensorFlow2. Unaligned ancestrally reconstructed sequences were tokenized into characters 1 through to 20, with each amino acid having a unique token, and padded at the N-terminus with a 0 pad character to the maximum sequence length (L) in the dataset, such that the cardinality of the dataset was constant. Padded and tokenized input arrays were then embedded in a 10-dimensional embedding layer before being input to two stacked bi-directional (bi) LSTMs with L cells, a hidden dimension of 64 in each direction (total embedding dimension of 128) and a dropout proportion of 0.1. Multiplicative self-attention was additionally implemented between the two stacked LSTMs. The last hidden layer of the biLS™, with cardinality of 128 was then fed into a dense layer with output dimension 21, which was softmaxed to a probability distribution over 21 categories. The LASR model was trained with a masked token objective, in which each token of each sequence is masked by a designated mask character and the model is trained to predict the identity of the masked character from the surrounding sequence context using a categorical cross-entropy loss function. This model was trained for 15 epochs using a batch size of 128 from Xavier uniform initialized weights and zero ininitialised biases. Once trained, information-rich vector embeddings of sequences were produced as the final hidden layer of the biLS™.

### Example 3: Generating Result Sequences

The processing device of Examples 1 and 2 uses the subset of the 20,000 evolved protein sequences of Example 2 to generate a plurality of result sequences. The plurality of result sequences is generated based on 95% redundancy threshold with respect to the test protein sequence of Example 1. The 20,000 evolved protein sequences are compared to the test protein sequence, and 18,000 protein sequences that do not have 95% sequence identity to the test protein sequence, and therefore, do not meet the 95% redundancy threshold, are removed. The processing device then provides the 2,000 remaining result sequences to an associated device.

### Example 4: Generating Result Sequences Based on a Test Sequence

The processing device of Examples 1, 2, and 3 with the trained LSTM machine learning model receives a new test protein sequence and applies a masked portion to the new test protein sequence. The processing device generates a plurality of 10,000 protein sequences phylogenetically related to the new test protein sequence using maximum likelihood ancestral sequence reconstruction. The trained LSTM machine learning model then generates a plurality of 16,000 evolved protein sequences by making additions, deletions, and/or substitutions to the 10,000 protein sequences. The 16,000 evolved protein sequences are then compared to the new test protein sequence, and 14,500 evolved protein sequences that do not meet a 95% redundancy threshold. The remaining 1,500 result sequences are then provided to an associated device.

### Example 5: Generating Evolved Sequences Using a Transformer Model

The processing device of Example 1 inputs the plurality of 10,000 protein sequences phylogenetically related to the test protein sequence of Example 1 to a Transformer machine learning model configured to learn contextual information relating to the protein sequence plurality. This is done by randomly masking a fraction of amino acids in each protein sequence in the plurality of protein sequences and training the model to predict which amino acids were masked. The processing device then receives the predictions from the transformer model as feedback to further train the transformer model by adjusting the parameters of the machine learning model.

The Transformer machine learning model was produced using PyTorch and TensorFlow2. Unaligned ancestrally reconstructed sequences were tokenized into characters 1 through to 20, with each amino acid having a unique token, and padding at the N-terminus with a 0 pad character to the maximum sequence length (L) in the dataset, such that the cardinality of the dataset was constant. Padded and tokenized input arrays were then embedded in a 128-dimensional embedding layer and added to a sinusoidal positional embedding of the same size. Then, dropout with a probability of 0.1 was applied before the embeddings were fed into 6 layers of 512-dimensional transformer encoder blocks with 4-headed multi-attention and a feed-forward fully connected layer. The subsequent output was then fed through a time-distributed fully-connected output layer to produce an array representing the probability of each amino acid at each site of the protein sequence. These probabilities were used to train the model to predict the identity of the masked character from the surrounding sequence context using a categorical cross-entropy loss function. This model was trained for 10 epochs using a batch size of 32 from Xavier uniform initialized weights and zero in initialized biases. Once trained, information-rich vector embeddings of sequences were produced as the final output of the transformer encoder blocks after undergoing mean average pooling across sequence length to produce 128-dimension sequence representations.

### Example 6: Generating Result Sequences

The processing device of Examples 1 and 5 uses the subset of the output embeddings of sequences of Example 5 to generate a plurality of result sequences. The plurality of result sequences is generated based on 95% redundancy threshold with respect to the test protein sequence of Example 1. The embeddings of sequences are compared to the test protein sequence, and 90% of the embeddings of sequences were found to have 95% sequence identity to the test protein sequence, and therefore, are removed. The processing device then provides the remaining embeddings of sequences to an associated device.

### Example 7: Generating Result Sequences Based on a Test Sequence

The processing device of Examples 1, 5, and 6 with the trained Transformer machine learning model receives a new test protein sequence and applies a masked portion to the new test protein sequence. The processing device generates a plurality of protein sequences phylogenetically related to the new test protein sequence using maximum likelihood ancestral sequence reconstruction. The trained Transformer machine learning model then generates a plurality of evolved protein sequences by making additions, deletions, and/or substitutions to the protein sequences. The evolved protein sequences are then compared to the new test protein sequence, and evolved protein sequences that do not meet a 95% redundancy threshold are removed. The remaining result sequences are then provided to an associated device.

### Example 8: Assessing Schemes for LASR

The performance of different embedding schemes was assessed for LASR as well as other state-of-the-art protein language models for the processing device of Examples 1-7. The schemes were ESM-1b, ESM-2, ProtTrans, and UniRep. To ascertain the impact of using ancestral sequence reconstructed sequences rather than extant sequences for training, a model of the same architecture as the model used for LASR was trained using the same scheme on the same amount of protein sequences as the model used for LASR.

Comparisons were made by first developing the best regression model possible for each representation scheme. Regression models were trained on experimentally determined catalytic efficiency values for 136 PTE variants. Using the Python package scikit-learn (version 1.2.1), a Bayesian Optimization approach was conducted to determine the best regression model and corresponding hyperparameter set for each embedding scheme. Bayesian optimization was conducted with an Expected Improvement acquisition function (ξ=0.01) on 5 initial random hyperparameter sets followed by 100 iterations of hyperparameter selection and testing. Hyperparameters were validated and selected to minimize the average mean absolute error over a 3-fold data split with a 20% pseudo-testing hold-out data set. Model architectures and hyperparameter ranges are seen in Table 1, which illustrates models and hyperparameter spaces used for model optimization and selection.

| Model | Search Hyperparameters |
|---|---|
| Random Forest (RF) | Estimators = [50, . . . , 1000] |
| | Maximum depth = (1, 10) |
| | Maximum features = [log2, sqrt, all] |
| | Minimum samples for split = (1e−4, 1) |
| | Minimum samples in leaf = (1e−4, 1) |
| Gradient Boosted Tree | Estimators = [1050 . . . , 1000] |
| (GBT) | Maximum depth = (1, 10) |
| | Maximum features = [log2, sqrt, all] |
| | Minimum samples for split = (1e−4, 1) |
| | Minimum samples in leaf = (1e−4, 1) |
| | Learning rate = (1e−4, 0.5) |
| | Subsample = (1e−4, 1) |
| Support Vector | Epsilon = (1e−4, 0.5) |
| Machine (SVM) | C = (1e−4, 100) |
| Gaussian Process (GP) | Alpha = (1e−4, 0.5) |

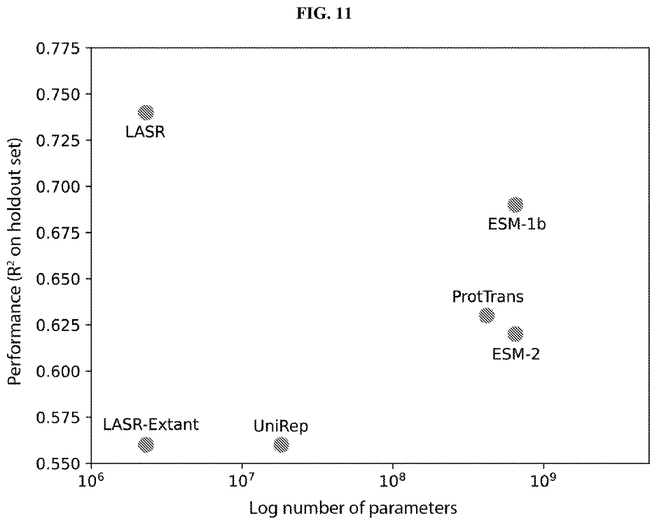

Protein variants that were in between the mutation pathway of the 136 training variants, but that had not previously been evaluated, were produced and the catalytic efficiency for each variant was experimentally determined for use as an unseen testing dataset to evaluate the performance of models trained on each embedding scheme. For each embedding scheme, the best model architecture was Random Forests (RFs), and hence, RFs were used to predict performance on the testing dataset. A coefficient of determination (R2) for each embedding scheme when using the optimized regression model is seen in FIG. 11 against the size of the embedding model (where the size was the logarithm of the number of parameters). When making this comparison and excluding observations related to using LASR, a trend between embedding model size and subsequent regression performance is observed, where larger models lead to higher regression performance.

LASR methods, however, are a clear outlier to this trend, where a smaller, light-weight embedding model results in superior regression performance. However, when an equivalent LASR model is developed using only extant sequences, the resulting performance drops, indicating that the use of ancestrally reconstructed sequences specifically allows the small architecture to capture the information that is required for improved regression performance.

A small sized embedding model such as those in LASR methods allows for both quicker training and use. To compare how the use of LASR over the next top-performing embedding scheme (ESM-1b) impacts their use for screening possible sequences, an in silico directed evolution experiment was conducted. Embedding schemes and corresponding RFs were used towards the in silico evolution of PTE with identical protocols. Ancestrally reconstructed sequences were used to determine a list of plausible mutations at each site in the 136 PTE training sequences. Sequences consisting of each possible single mutation were made, duplicate sequences were removed, and 250 PTE variants with the highest predicted catalytic efficiency were then used for the next generation. This was repeated for 15 generations. In total, almost 250,000 sequences were embedded and had their activity predicted. The total wall-time for this experiment (FIG. 12) was approximately 2 minutes 9 seconds when using LASR compared to approximately 2 hours 12 minutes 46 seconds when using ESM-1b.

While preferred embodiments of the present subject matter have been shown and described herein, it will be obvious to those skilled in the art that such embodiments are provided by way of example only. It is not intended that the subject matter described herein be limited by the specific examples provided within the specification. While the present subject matter has been described with reference to the aforementioned specification, the descriptions and illustrations of the embodiments herein are not meant to be construed in a limiting sense. Numerous variations, changes, and substitutions will now occur to those skilled in the art without departing from the subject matter described herein. Furthermore, it shall be understood that all aspects of the present subject matter are not limited to the specific depictions, configurations or relative proportions set forth herein which depend upon a variety of conditions and variables. It should be understood that various alternatives to the embodiments of the subject matter described herein may be employed in practice. It is therefore contemplated that the present subject matter shall also cover any such alternatives, modifications, variations or equivalents. Itis intended that the following claims define the scope of the present subject matter and that methods and structures within the scope of these claims and their equivalents be covered thereby.

1.-108. (canceled)

109. A method of generating a plurality of biological sequences, comprising:
receiving at least one test biological sequence;
generating a plurality of reconstructed sequences by applying an algorithm comprising ancestral sequence reconstruction (ASR) to the at least one test biological sequence;
selecting a plurality of refined reconstructed sequences from the reconstructed sequences;
generating, using an evolution machine learning model, a plurality of evolved sequences from the refined reconstructed sequences, wherein at least one of the evolved biological sequences is derived from one of the refined reconstructed sequences with one or more evolutions;
selecting a plurality of refined evolved sequences from the evolved sequences;
refining the refined evolved sequences based on one or more parameters to generate a plurality of result sequences; and
providing the result sequences.

110. (canceled)

111. The method of claim 109, further comprising:
receiving feedback based on the result sequences; and
training the evolution machine learning model based on the feedback.

112.-113. (canceled)

114. The method of claim 109, wherein the one or more parameters are received from a device associated with a user.

115. The method of claim 109, wherein the one or more parameters comprises at least one of:
a sequence redundancy threshold, wherein the redundancy threshold is about 50% to about 99%, or about 50%, or about 70% to about 99%, or about 70%, or about 90%, 91%, 92%, 93%, 94%, 95%, 96%, 97%, 98%, or 99%; or
one or more biological sequences.

116. The method of claim 109, wherein the evolution machine learning model generates the evolved sequences based at least in part on:
a first list containing a distribution of filtered sequence lengths of the evolved sequences per percent identity redundancy based on a redundancy threshold; or
a second list containing a number of biological sequences of the evolved sequences per percent identity based on the redundancy threshold.

117. The method of claim 109, further comprising storing the reconstructed sequences, the evolved sequences, and/or the result sequences in a database.

118. The method of claim 109, further comprising displaying the result sequences.

119. (canceled)

120. The method of claim 109, wherein the one or more evolutions comprise at least one addition, deletion, or substitution.

121. The method of claim 109, wherein the second plurality of biological evolved sequences comprises:
a plurality of accession numbers corresponding to the sequences of the evolved sequences;
a plurality of accession numbers associated with experimental validation, wherein the plurality of accession numbers corresponds to a first subset of the evolved sequences; and/or
a plurality of accession numbers associated with solved crystal structures, wherein the plurality of accession numbers corresponds to a second subset of the evolved sequences.

122. The method of claim 109, wherein the result sequences comprise a first subset of the reconstructed sequences and a second subset of the evolved sequences.

123. The method of claim 109, wherein the result sequences have fewer sequences than the evolved sequences.

124.-130. (canceled)

131. The method of claim 109, wherein the evolution machine learning model comprises a transformer model or a long short-term memory (LSTM) recurrent neural network model.

132. (canceled)

133. The method of claim 109, wherein the reconstructed sequences comprise at least ten thousand different biological sequences.

134. The method of claim 133, wherein generating the reconstructed sequences comprises refining the test sequences to no more than five hundred different biological sequences.

135.-138. (canceled)

139. The method of claim 109, wherein refining the evolved sequences based on one or more parameters to generate the result sequences comprises applying a refinement machine learning model to the evolved sequences to optimize the evolved sequences based on one or more indications.

140. The method of claim 139, wherein the one or more indications comprises one or more of:
robustness of at least one biological sequence of the evolved sequences;
stability of at least one biological sequence of the evolved sequences;
percent identity of at least one biological sequence of the evolved sequences to a selected biological sequence;
activity of at least one biological sequence of the evolved sequences;
expression of at least one biological sequence of the evolved sequences.

141.-144. (canceled)

145. A system, comprising:
one or more computer processors; and
a memory comprising executable instructions which, when executed by the one or more processors, cause the system to implement the method of claim 109.

146.-180. (canceled)

181. At least one non-transitory, computer-readable medium comprising machine executable code that, upon execution by one or more computer processors, implements the method of claim 109.

182.-216. (canceled)

217. The method of claim 109, wherein the algorithm comprises:
performing a plurality of independent heuristic tree searches in parallel to reconstruct phylogenetic topologies from the at least one test biological sequence,
filtering ones of these reconstructed phylogenetic topologies that are statistically equivalent by the approximately unbiased (AU) test to generate a pool of distinct phylogenies, and
using the pool of distinct phylogenies to reconstruct the reconstructed sequences by Ancestral Sequence Reconstruction (ASR).

218. The method of claim 217, wherein the one or more parameters comprise at least one of:
a sequence redundancy threshold, wherein the redundancy threshold is about 50% to about 99%, or about 50%, or about 70% to about 99%, or about 70%, or about 90%, 91%, 92%, 93%, 94%, 95%, 96%, 97%, 98%, or 99%; or
one or more biological sequences.
